(19) United States
(12) Patent Application Publication
(10) Pub. No.: US 2020/0283834 A1
(43) Pub. Date: Sep. 10, 2020

(54) RAPID IN SITU DETECTION OF DNA AND RNA

(71) Applicant: THE REGENTS OF THE UNIVERSITY OF CALIFORNIA, Oakland, CA (US)

(72) Inventors: Bo HUANG, San Francisco, CA (US); Juan GUAN, San Francisco, CA (US)

(21) Appl. No.: 16/651,932

(22) PCT Filed: Oct. 8, 2018
(86) PCT No.: PCT/US2018/054881

§ 371 Date: Mar. 27, 2020

Related U.S. Application Data
(60) Provisional application No. 62/594,188, filed on Dec. 4, 2017.
(60) Provisional application No. 62/568,964, filed on Oct. 6, 2017.

(51) Int. Cl. C12Q 1/6841

(52) U.S. Cl. CPC C12Q 1/6841

(57) ABSTRACT

DNA and RNA fluorescence in situ hybridization (FISH) is a widely used method to analyze the copy number and spatial localization of specific DNA and RNA sequences in the nuclear. Here we introduce an approach to achieve highly multiplexed sequential DNA or RNA FISH at a fast speed. In one approach, one first performs one-color live imaging using the CRISPR imaging method for multiple genomic loci and then uses sequential rounds of DNA FISH to determine the loci identity. The FISH protocol described herein has been developed so that each round of hybridization is complete in 1 min. for example demonstrating the identification of 7 genomic elements and the capability to sustain reversible staining and washing for example for up to 20 rounds. In another approach, one can profile the gene expression pattern of a single cell using rapid and sequential RNA FISH. Each round of RNA FISH is complete in 5-10 min.

### CROSS-REFERENCE TO RELATED PATENT APPLICATIONS

The present patent application claims benefit of priority to U.S. Provisional Patent Application No. 62/568,964, filed Oct. 6, 2017 and U.S. Provisional Patent Application No. 62/594,188, filed Dec. 4, 2017, each of which are incorporated by reference

### STATEMENT AS TO RIGHTS TO INVENTIONS MADE UNDER FEDERALLY SPONSORED RESEARCH AND DEVELOPMENT

This invention was made with government support under grant no. R33 EB019784 awarded by the National Institutes of Health. The government has certain rights in the invention.

### BACKGROUND OF THE INVENTION

In fixed systems, highly multiplexed fluorescence in situ hybridization for both RNA (Lubeck, E. 2014. Nat Methods 11:360-361; Coskun, A. F., and L. Cai. 2016. Nat Methods 13:657-660; Chen, K. H. 2015. Science 348:aaa6090) and DNA (Wang, S. 2016. Science 353:598-602) has been reported by sequentially applying and imaging different probes according a prearranged code, followed by removing of the probe signal by photobleaching, photocleavage or enzymatic digestion before applying subsequent rounds of probes. Tens or even hundreds of DNA or RNA species can be distinguished in this way. However, each round of staining and de-staining typically takes tens of minutes to hours, which substantially limits the practical application of these methods.

### BRIEF SUMMARY OF THE INVENTION

It is a surprising and unexpected discovery of the present disclosure that in situ hybridization (ISH) and detection of genomic DNA fragments, as well as RNA, can be completed within a few minutes, making multiplexed and sequential ISH practical. Certain aspects of the presently described technology are believed to contribute to this surprising success.

For instance, conventionally, target RNA sequences do not require denaturing before hybridization. The present inventors, however, discovered that denaturation prior to hybridization greatly expedites the hybridization efficiency. Likewise, for DNA ISH, when the denaturation is carried out to a suitable greater extent as compared to the conventional approach (e.g., by increasing the temperature and/or duration), the hybridization efficiency is also improved.

The modified hybridization conditions for both DNA and RNA ISH are also believed to have contributed to the improvement of ISH efficiency. Conventionally, long oligonucleotides are used for hybridization to achieve high specificity and strong signal. In certain embodiments of the present technology, the oligonucleotides are no longer than half of the length of oligonucleotides used conventionally. Conventionally, large molecules such as salmon sperm and dextran sulfate are added into the hybridization buffer to suppress nonspecific binding or accelerate the hybridization of long oligonucleotides. In certain embodiments of the present technology, however, the hybridization buffer includes no, or a relatively small amount of such large molecules.

In accordance with one embodiment of the present disclosure, therefore, provided is a method for conducting in situ detection of a genomic sequence in a cell. In some embodiments, the method entails fixing and permeabilizing the cell, denaturing genomic sequences in the cell, contacting the denatured genomic sequences with an oligonucleotide probe less than 150 nucleotides in length (or alternatively less than 140, 130, 120, 110, 100, 90, 80, 70, 60, 50 nucleotides in length) in a hybridization buffer that does not include more than about 10% (w/v of hybridization buffer; or alternatively does not include more than about 9%, 8%, 7%, 6%. 5%, 4%, 3%, 2%, 1%, 0.9%. 0.8%, 0.7%, 0.6%, 0.5%, 0.4%. 0.3%, 0.2%, 0.1%, 0.05%, or 0.01%) of molecules having a molecular weight of greater than 500,000 daltons (or alternatively greater than 400,000 Da, 200,000 Da, 100,000 Da, 80,000 Da, 50,000 Da, 30,000 Da, 20,000 Da, 10,000 Da, 8,000 Da, 7,000 Da, 5.000 Da, 4000 Da, 3,000 Da, 2,000 Da, 1,500 Da, 1,000 Da, 900 Da, 800 Da. 700 Da. 600 Da, 500 Da, 400 Da, 300 Da, 200 Da, 150 Da or 100 Da), and detecting a genomic sequence hybridized to the probe.

In accordance with one embodiment of the present disclosure, therefore, provided is a method for conducting in situ detection of a RNA sequence in a cell. In some embodiments, the method entails fixing and permeabilizing the cell, denaturing RNA sequences in the cell, contacting the denatured RNA sequences with an oligonucleotide probe less than 150 nucleotides in length (or alternatively less than 140, 130, 120, 110, 100, 90, 80, 70, 60, 50 nucleotides in length) in a hybridization buffer that does not include more than about 20% (w/v of hybridization buffer, or alternatively does not include more than about 18%, 15%, 12%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%. 0.05%, or 0.01%) of molecules having a molecular weight of greater than 500,000 daltons (or alternatively greater than 400,000 Da, 200,000 Da, 100,000 Da. 80,000 Da, 50,000 Da, 30,000 Da, 20,000 Da, 10,000 Da, 8,000 Da, 7,000 Da, 5,000 Da, 4000 Da, 3,000 Da, 2,000 Da, 1,500 Da, 1,000 Da, 900 Da, 800 Da, 700 Da, 600 Da, 500 Da, 400 Da, 300 Da, 200 Da, 150 Da or 100 Da), and detecting a RNA sequence hybridized to the probe.

In some embodiments, the composition of the hybridization buffer reflects the hybridization conditions. In other words, the hybridization is carried out without the presence of more than about 20%, 18%, 15%, 12%, 10%, 9%, 8%, 7%, 6%, 5%, 5%, 4%, 3%, 2%, 1%, 0.9%, 0.8%, 0.7%. 0.6%. 0.5%, 0.4%, 0.3%, 0.2%, 0.1%. 0.05%, or 0.01% w/v) of molecules having a molecular weight of greater than 500,000 daltons (or alternatively greater than 400,000 Da, 200,000 Da, 100,000 Da, 80,000 Da, 50,000 Da, 30,000 Da, 20,000 Da, 10,000 Da, 8,000 Da, 7,000 Da, 5,000 Da, 4000 Da, 3,000 Da, 2,000 Da, 1,500 Da, 1,000 Da, 900 Da, 800 Da, 700 Da, 600 Da. 500 Da. 400 Da. 300 Da. 200 Da, 150 Da or 100 Da).

In some embodiments, the hybridization is carried out for no more than about 15 minutes. In some embodiments, the hybridization is carried out for no more than about 14, 13, 12, 11, 10, 9, 8, 7, 6, 5, 4, 3, 2, or 1 minutes. In some embodiments, the detection is made following the hybridization within the prescribed limited time.

In some embodiments, the denaturation step is carried out at 80° C. for 5-10 min (or alternatively 700° C. for 10-30 min, 72° C. for 10-20 min, 75° C. for 5-15 min, 80° C. for 10-20 min, 85° C. for 3-15 min, 90° C. for 2-12 min, 95° C. for 1-10 min, 100° C. for 1-10 min).

In some aspects, methods of in situ detecting nuclear DNA sequences in eukaryotic cells are provided. In some embodiments, the methods comprise,

fixing the eukaryotic cells with formaldehyde for a duration shorter than 10 minutes;
permeabilizing the eukaryotic cells using a lipid-dissolving solvent;
denaturing nuclear DNA in the eukaryotic cells that have been fixed and permeabilized, wherein the denaturing comprises incubating the eukaryotic cells in a sufficient amount of a DNA strand interaction-weakening agent under sufficiently high heating conditions and for a sufficient time to denature the nuclear DNA;
contacting the cells having denatured DNA with a solution comprising a first set (e.g., one or more than one, e.g., 10-1000, e.g., 10-200, 12-100) of labelled single-stranded oligonucleotides (e.g., of 13-55, or 13-40 nucleotides in length), wherein all components of the solution at a concentration of at least 0.1%, 1%, or 5% (w/w) have a molecular weight of less than 10,000 daltons;
hybridizing at least some of the oligonucleotides to the nuclear DNA in the cells;
washing away non-hybridized labelled oligonucleotides; and
detecting signal from labelled oligonucleotides hybridized to nuclear DNA in the eukaryotic cells in situ, thereby in situ detecting nuclear DNA sequences in the eukaryotic cells.

In some embodiments, the hybridized oligonucleotides are removed and the method is repeated with different labeled oligonucleotide. In some embodiments, the method further comprises:

removing hybridized oligonucleotides from the eukaryotic cells; and then
contacting the cells having denatured DNA with a solution comprising a second set (10-1000, e.g., 10-200, 12-100) of labelled single-stranded oligonucleotides (e.g., of 13-55 or 13-40 nucleotides in length), wherein all components of the solution at a concentration of at least 0.1%, 1%, or 5% (w/w) have a molecular weight of less than 1000 or 10,000 daltons;
hybridizing at least some of the second set of oligonucleotides to the nuclear DNA in the cell;
washing away nonhybridized labelled oligonucleotides; and
detecting signal from second set of labelled oligonucleotides hybridized to nuclear DNA in the eukaryotic cells in situ.

In some embodiments, the lipid-dissolving solvent is methanol, ethanol, methanol and ethanol, or acetone. In some embodiments, the DNA strand interaction-weakening agent is formamide or ethylene carbonate.

In some embodiments, the first set of oligonucleotides comprise at least 10, 20, 30, 40, 50 or more oligonucleotides each having different sequences.

In some embodiments, the denaturing comprises incubating the eukaryotic cells in 70-95% formamide for at least 5 or 10 minutes or for 1-20 minutes, e.g., 5-15 minutes.

In some embodiments, the method takes less than 45, 30, 20, or 15 minutes from the fixing to the detecting. In some embodiments, the time between the hybridizing and the washing is less than 10 (e.g., less than 5 or 3) minutes.

In some embodiments, the cells are not contacted with more than 0.1%, 1%, or 5% (w/wv) dextran sulfate. In some embodiments, the cells are not contacted with dextran sulfate.

In some embodiments, the label is fluorescent.

In some embodiments, the hybridizing comprises contacting the cells with a buffer comprising 0-90% (e.g., zero, or 1-90%, or 10-90%) formamide for less than 10 (e.g., 5, 4, 3, 2) minutes.

In some embodiments, the fixing occurs before the permeabilizing. In some embodiments, the permeabilizing occurs before the fixing.

In some embodiments, labelled single-stranded oligonucleotides are between 15-35 nucleotides long.

In some embodiments, the eukaryotic cells are mammalian cells.

In other aspects, methods of in situ detecting RNA sequences in eukaryotic cells are provided. In some embodiments, the methods comprise.

fixing the eukaryotic cells with formaldehyde for a duration shorter than 10 minutes;
permeabilizing the eukaryotic cells using a lipid-dissolving solvent;
contacting the cells having RNA with a solution comprising a first set (e.g., one or more than one, e.g., 10-1000, e.g., 10-200, 12-100) of labelled single-stranded oligonucleotides (e.g., of 13-55, or 13-40 nucleotides in length), wherein all components of the solution at a concentration of at least 0.1%, 1%, or 5% (w/w) have a molecular weight of less than 1000 or 10,000 daltons;
hybridizing at least some of the oligonucleotides to the RNA in the cells;
washing away non-hybridized labelled oligonucleotides; and
detecting signal from labelled oligonucleotides hybridized to RNA in the eukaryotic cells in situ, thereby in situ detecting RNA sequences in the eukaryotic cells. Additional embodiments for detection of RNA can include those described herein for DNA detection.

In some embodiments, the method further comprises:

removing hybridized oligonucleotides from the eukaryotic cells; and then
contacting the cells having RNA with a solution comprising a second set (e.g., one or more than one, e.g., 10-1000, e.g., 10-200, 12-100) of labelled single-stranded oligonucleotides (e.g., of 13-55, or 13-40 nucleotides in length), wherein all components of the solution at a concentration of at least 0.1%, 1%, or 5% (w/w) have a molecular weight of less than 1000 or 10,000 daltons;
hybridizing at least some of the oligonucleotides from the second set to the RNA in the cells;
washing away non-hybridized labelled oligonucleotides; and
detecting signal from labelled oligonucleotides from the second set hybridized to RNA in the eukaryotic cells in situ.

In some embodiments, the method further comprises denaturing RNA in the cell after the fixing and permeabilizing and before the contacting In some embodiments, the denaturing comprises incubating the eukaryotic cells in a sufficient amount of a nucleic acid strand interaction-weakening agent under sufficiently high heating conditions and for a sufficient time to denature RNA in the eukaryotic cells. In some embodiments, the denaturing comprises incubating the eukaryotic cells in 70-95% formamide for at least 5 or 10 minutes.

In some embodiments, the lipid-dissolving solvent is methanol, ethanol, methanol and ethanol, or acetone.

In some embodiments, the nucleic acid strand interaction-weakening agent is formamide or ethylene carbonate.

In some embodiments, the first set of oligonucleotides comprise at least 30 oligonucleotides each having different sequences.

In some embodiments, the method takes less than 45, 30, 20, or 15 minutes (and optionally more than 5 or 10 minutes) from the fixing to the detecting. In some embodiments, the time between the hybridizing and the washing is less than 10 (e.g., less than 5 or 3) minutes.

In some embodiments, the cells are not contacted with more than 0.1% dextran sulfate. In some embodiments, the cells are not contacted with dextran sulfate.

In some embodiments, the label is fluorescent.

In some embodiments, the hybridizing comprises contacting the cells with a buffer comprising 0-90% formamide for less than 10 (e.g., 5, 4, 3, 2) minutes.

In some embodiments, the hybridizing comprises contacting the cells with a buffer comprising 0-90% formamide for less than 10 (e.g., 5, 4, 3, 2) minutes.

In some embodiments, the fixing occurs before the permeabilizing In some embodiments, the permeabilizing occurs before the fixing.

In some embodiments, labelled single-stranded oligonucleotides are between 15-35 nucleotides long.

In some embodiments, the eukaryotic cells are mammalian cells.

Further aspects include the following

Aspect 1. A method of in situ detecting cellular DNA in a biospecimen, the method comprising,

fixing and permeabilizing the cells in the biospecimen,
denaturing DNA in the cells, wherein the denaturing comprises incubating the cells in a sufficient amount of a DNA strand interaction-weakening agent under sufficiently high heating conditions and for a sufficient time;
contacting the denatured cellular DNA with a hybridization solution comprising a first set (e.g., one or more than one, e.g., 10-1000, e.g., 10-200, 12-100) of labelled single-stranded oligonucleotides of less than 150 nucleotides in length (e.g., of 13-55, 30-70, 50-100, or 70-120 nucleotides in length), and a hybridization buffer;
and
detecting the oligonucleotides hybridized to the cellular DNA in situ.

Aspect 2. The method of Aspect 1 further comprising:

removing hybridized oligonucleotides from the cells; and then
contacting the cells having denatured DNA with a solution comprising a second set of labelled single-stranded oligonucleotides of less than 150 nucleotides in length:
hybridizing at least some of the second set of oligonucleotides to the DNA in the cell;
washing away nonhybridized labelled oligonucleotides; and
detecting signal from second set of labelled oligonucleotides hybridized to DNA in the cells in situ.

Aspect 3. The method of Aspect 1 or 2, wherein the fixation and permeabilizing agent is formaldehyde, acetic acid, methanol, ethanol, methanol and ethanol, or acetone, or combination of two or more of them.

Aspect 4. The method of Aspect 1 or 2, wherein the DNA strand interaction-weakening agent is formamide or ethylene carbonate.

Aspect 5. The method of Aspect 1 or 2, wherein the first set of oligonucleotides comprise at least two oligonucleotides each having different sequences.

Aspect 6. The method of Aspect 1, wherein the denaturing temperature is 70° C. or higher.

Aspect 7. The method of Aspect 1 or 2, wherein the denaturing comprises incubating the cells in 70-95% formamide for at least 5 or 10 minutes.

Aspect 8. The method of Aspect 1, wherein the method takes less than 45, 30, 20, or 15 minutes from the fixing to the detecting.

Aspect 9. The method of Aspect 1 and 2, wherein the time between the hybridizing and the following removing of oligonucleotides is less than 15 (e.g., less than 10, 5 or 3) minutes.

Aspect 10. The method of Aspect 1 or 2, wherein the cells are not contacted with more than 10% (w/v of hybridization solution) dextran sulfate with molecular weight more than 10,000 daltons.

Aspect 11. The method of Aspect 1, wherein the cells are not contacted with dextran sulfate.

Aspect 12. The method of any of Aspects 1-11, wherein the label is fluorescent.

Aspect 13. The method of Aspect 1, wherein the hybridizing comprises contacting the cells with a buffer comprising 0-90% formamide for less than 10 (e.g., 5, 4, 3, 2) minutes.

Aspect 14. The method of Aspect 2, wherein the hybridizing comprises contacting the cells with a buffer comprising 0-90% formamide for less than 10 (e.g., 5, 4, 3, 2) minutes.

Aspect 15. The method of Aspect 1 or 2, wherein labelled single-stranded oligonucleotides are between 10-35 nucleotides long.

Aspect 16. The method of any of Aspects 1-15, wherein the cells are eukaryotic cells or prokaryotic cells.

Aspect 17. A method of in situ detecting RNA sequences in cells, wherein the method comprises,

fixing and permeabilizing the cells;
denaturing the RNA in the cells at a temperature above room temperature;
contacting the cells having RNA with a solution comprising a first set (e.g., one or more than one, e.g., 10-1000, e.g., 10-200, 12-100) of labelled single-stranded oligonucleotides of less than 150 nucleotides in length (e.g., of 13-55, 30-70 or 50-100 nucleotides in length), and a hybridization buffer,
hybridizing at least some of the oligonucleotides to the RNA in the cells;
washing away non-hybridized labelled oligonucleotides; and detecting signal from labelled oligonucleotides hybridized to RNA in the cells in situ, thereby in situ detecting RNA sequences in the cells.

Aspect 18. The method of Aspect 17, further comprising:

removing hybridized oligonucleotides from the cells by a heated buffer above room temperature (i.e., 23° C.); and then
contacting the cells having RNA with a solution comprising a second set (e.g., one or more than one. e.g., 10-1000, e.g., 10-200, 12-100) of labelled single-stranded oligonucleotides (e.g., of 13-55, or 30-70 nucleotides in length), and a hybridization buffer,
hybridizing at least some of the oligonucleotides from the second set to the RNA in the cells;
washing away non-hybridized labelled oligonucleotides; and
detecting signal from labelled oligonucleotides from the second set hybridized to RNA in the cells in situ.

Aspect 19. The method of Aspects 17 and 18, wherein cells are attached onto a scaffold that is optical transparent;

Aspect 20. The method of Aspects 17-19, wherein cells are attached onto a glass coverslip with a thickness less than 1 mm.

Aspect 21. The method of Aspect 17, wherein the denaturing comprises incubating the cells in a sufficient amount of a nucleic acid strand interaction-weakening agent under sufficiently high heating conditions and for a sufficient time to denature RNA in the cells.

Aspect 22. The method of Aspect 17, wherein the fixation and permeabilization reagent is formaldehyde, acetic acid, methanol, ethanol, methanol and ethanol, or acetone, or combination of two or more of them.

Aspect 23. The method of Aspect 18, wherein the washing buffer to remove hybridized oligonucleotides has a temperature at 50° C. or higher.

Aspect 24. The method of Aspects 17 and 18, wherein the nucleic acid strand interaction-weakening agent is formamide or ethylene carbonate.

Aspect 25. The method of Aspects 17 and 18, wherein the first set of oligonucleotides comprise at least 2 oligonucleotides each having different sequences.

Aspect 26. The method of Aspects 17 and 18, wherein the denaturing comprises incubating the cells in 70-95% formamide for at least 5 or 10 minutes.

Aspect 27. The method of Aspect 17, wherein the method takes less than 60, 30, 20, or 15 minutes (and optionally more than 5 or 10 minutes) from the fixing to the detecting.

Aspect 28. The method of Aspect 17, wherein the time between the hybridizing and the washing is less than 30 (e.g., less than 15 or 5) minutes.

Aspect 29. The method of Aspects 17 and 18, wherein the cells are not contacted with more than 20% (w/v of hybridization solution) dextran sulfate with molecular weight more than 500,000 daltons.

Aspect 30. The method of Aspects 17 and 18, wherein the cells are not contacted with dextran sulfate.

Aspect 31. The method of any of Aspects 17-30, wherein the label is fluorescent.

Aspect 31a. The method of Aspects 17 or 18, wherein the hybridizing comprises contacting the cells with a buffer comprising 0-90% formamide for less than 10 (e.g., 5, 4, 3, 2) minutes.

Aspect 32. The method of Aspects 17 or 18, wherein labelled single-stranded oligonucleotides are between 10-35 nucleotides long.

Aspect 33. The method of any of Aspects 17-32, wherein the cells are eukaryotic cells or prokaryotic cells.

Aspect 34. A hybridization buffer (oligo probes excluded) for in situ detecting DNA or RNA in cells, wherein all components of the hybridization solution at a concentration of at least 0.1%, 1%, or 5% (w/v) have a molecular weight of less than 10,000 daltons.

Aspect 35. The hybridization buffer of Aspect 34, wherein all components with a molecular weight of less than 5,000 daltons.

Aspect The hybridization buffer of Aspect 34 comprises formamide, NaCl, sodium citrate and H₂O.

Other embodiments are described elsewhere herein.

### BRIEF DESCRIPTION OF THE DRAWINGS

FIG. 1 Schematic of correlative CRISPR imaging and sequential DNA FISH. Live cells are first imaged in time-lapse mode to acquire dynamics information. Multiple genomic loci are simultaneously imaged without distinguishing their identities. Cells are fixed immediately after live imaging. Rapid sequential rounds of DNA FISH are performed afterwards. As probes specifically bound to a locus are introduced in each round, the identity of the locus is resolved by comparing the last frame of live image and fixed images.

FIG. 2 Rapid staining can be achieved with oligo DNA probes under optimized DNA FISH protocol. (a) Kinetics of the rapid DNA FISH staining. Intensity of FISH puncta for a tandemly repetitive sequence in the human genome is shown. Intensity is averaged over 300+ puncta and error bars denote standard deviation. (b) Intensity of nuclear background staining at different time points. (c) The signal-to-noise ratio of FISH puncta. (d) The efficiency of FISH staining, calculated as the ratio of observed to expected number of puncta. (e-h) Representative images at different time points of staining. Maximum intensity projection is shown for each 3D z-stack. (i-j) Representative images of two genomic regions denoted as NR1(b) and NR2 (c) of non-repetitive sequence labeled by tiling ˜200 probes over 40-kb region with a staining time of 2 min. See Materials and Methods section for details on probe sequence. Maximum intensity projection is shown for each 3D z-stack.

FIG. 3 Multiple genomic loci can be resolved through multiple sequential rounds of DNA FISH. (a-g) Chr13, Chr7, Chr3, Chr3*, Chr1, telomere and centromere are sequentially resolved in six rounds of staining of 1 min and wash of 0.5 min. Two sites 300 kb apart on chromosome 3 (denoted as Chr3 and Chr3*) are co-stained and distinguished in one round with two colors. Maximum intensity projection is shown for each 3D z-stack. The nucleus contour is denoted as dashed yellow line. (h) False color overlay of telomere (white) and centromere (blue) staining. (i) False color overlay of Chr13 (yellow). Chr7 (magenta). Chr3 (red). Chr3* (green), and Chr1 (cyan). (j) Overlay of all loci determined in the sequential DNA FISH. Color is the same as in h and i. Image area in a-j is 22 μm×22 μm. (k) Peak intensity value of 20 rounds of repeated staining of 1 min and washing of 0.5 min with nuclear background subtracted, averaged over 8 loci. Error bars denote standard deviation. The nuclear background (orange line) is calculated as the average intensity within a nuclear region minus the average intensity from the empty region without cells.

FIG. 4 Sequential rounds of DNA FISH resolve identities of loci imaged in live-cell mode. (a-c) Representative micrographs of a cell from time-lapse images. 8 spots corresponding to 4 genomic regions in a diploid RPE cell are seen. Green arrowhead highlights a pair of sister chromatids that are distinguished at the beginning of the live imaging and came closer than the diffraction limit at later time. Red arrowhead highlights another pair of sister chromatids that are distinct throughout the image acquisition with fluctuating separation. (d) Overlay of the cell image at the end of the live observation with the corresponding loci trajectories. Color denotes time in minutes. (e-h) FISH images in four sequential FISH rounds reveal the identities of the loci. (i-l) Overlay of live images at 25 min with DNA FISH images from each round shows faithful registration and negligible nuclear deformation. The FISH spots in each round are highlighted with arrows. (m-p) Four sequential DNA FISH rounds staining Chr13 show consistent image registration between rounds. All micrographs here show the same image area and scale bar in (a) is 5 μm.

FIG. 5 Simultaneous live CRISPR imaging of multiple genomic elements and cell cycle tracking. (a-d) Correlative imaging of CRISPR imaging and sequential DNA-FISH is performed on cells with Fucci cell cycle tracker at G1, onset of S, early S, and G2 phase respectively. The first three columns in live panel correspond to signals from Fucci cell tracker and the forth column in live panel shows CRISPR imaging where puncta are highlighted in circle. The first four columns in FISH panel are FISH images corresponding to four different genomic loci and the last columns in FISH panel shows all FISH puncta identified in sequential rounds in false-color. Nuclei are outlined with dotted line. (e) Schematic of Fucci cell cycle tracker.

FIG. 6 shows two example images of GAPDH and Neat1 RNA in human RPE cells as described in Example 2. (a) GAPDH gene expression detected by fluorophore labeled oligonucleotides in RPE cells. (b) Neat1 gene expression detected by fluorophore labeled oligonucleotides in RPE cells.

FIG. 7 RNA FISH are accelerated by heat denaturation. A set of 48 fluorophore labeled oligo probes with 20 nucleotides in length were used here to detect the gene expression of GAPDH in RPE cells. All images are shown at the same image contrast. (a): 37° C. hybridization for 5 min without pre-heat denaturation. (b): 50° C. denaturation for 10 min and then 37° C. hybridization for 5 min.(c): 70° C. denaturation for 10 min and then 37° C. hybridization for 5 min. (d): 80° C. denaturation for 10 min and then 37° C. hybridization for 5 min.

FIG. 8 RNA FISH at 37° C. without heat denaturation but in a simplified hybridization buffer only. The composition of the hybridization buffer is 10% formamide, 2×SSC. Fluorescent labeled oligo probes to detect GAPDH gene expression were used here. (a), 30 min hybridization at 37° C. without heat denaturation. (b), 60 min hybridization at 37° C. without heat denaturation.

FIG. 9 Rapid DNA FISH requires sufficient heat denaturation. (a) 70° C. 2 min denaturation; (b) 70° C. 5 min denaturation; (c) 75° C. 2 min denaturation: (d) 70° C. 10 min denaturation; (e) 80° C. 10 min denaturation; (f) 95° C. 3 min denaturation. HeLa cells are used here for staining and imaging.

FIG. 10 Rapid DNA FISH in white blood cells. (a) The same probe to target the repetitive sequence of telomere in Example 1 was used to detect telomere in human white blood cells. (b) The same probe to target the repetitive sequence of chromosome 1 in Example 1 was used to detect chromosome 1 in human white blood cells. (c) An oligo pool library with 1096 oligos were designed to target chromosome 19 in human white blood cells.

FIG. 11 Schematic of the 3D-printed stage adaptor to fit the 8-well imaging chamber (Lab-Tek) onto the microscope stage.

FIG. 12 Stage registration algorithm. See methods in Examples for details.

FIG. 13 Refined image registration algorithm to register loci features based on nuclear shape. See methods in Examples for details. Field of view is 30 μm×30 μm.

FIG. 14 The image registration algorithm remains robust when features overlap at a high density. The overlapping feature is generated by overlaying two less dense images.

FIG. 15 Human genome is abundant with tandemly repetitive sequences (TRS) potentially compatible with CRISPR imaging, about 100 loci per chromosome. The red bars denote the positions of these TRS site on the 24 human chromosomes.

FIG. 16 The efficiency of FISH staining is consistently almost 100%. The efficiency is calculated as the ratio of observed to expected number of FISH puncta. Error bars denote standard deviation.

FIG. 17 Examples of two oligo sequences staining human 5s DNA which has a copy number ranging from 35 to 175.

FIG. 18 Heating duration affects the accessibility of genome. The same cell shows 0, 1, and 2 spots after heating at 80 C for 0 min. 3 min, and 7 min respectively. Staining time is 2 min. A second stain after washing and removing the bound probes at each time point confirms the trend.

FIG. 19 (left) Apparent trajectories of loci reflecting the absolute displacement overlaid with live image at t=0 min. (Right) Adjusted trajectories of loci reflecting the relative displacement after subtracting the global movement of nucleus overlaid with live image at t=0 min. The relative displacement between loci is reduced in amplitude and randomized in direction compared to absolute displacement. The nucleus is the same as in FIG. 4.

FIG. 20 Two representative trajectories overlaid with the nuclei at t=60 min. Global movement of nuclei and stable relative position of loci is a common feature observed in live-cell imaging.

FIG. 21 (a) The scatter plot of loci position in fixed images compared to live-cell position set at the origin. (b-c) The histogram showing the error distribution in registration of loci position. The RMS error is ˜52 nm.

FIG. 22 (a) The scatter plot of loci position between two sequential DNA FISH hybridization rounds with the loci position in the first round set at the origin. (b-c) The histogram showing the error distribution in registration of loci position. The RMS error is ˜43 nm.

FIG. 23 (a) Principle component analysis of nuclear shape. The first two principal components correspond to the long and short axis of nuclei. θ characterize the nuclear orientation. (b-c) The histogram of long and short axis distribution in live-cell imaging respectively. (d-t) The histogram of the change between live-cell images and fixed images in long axis, short axis, and angle of rotation, respectively.

### DETAILED DESCRIPTION OF THE INVENTION

New rapid methods for in situ detection of DNA or RNA sequences (e.g., nuclear DNA for DNA FISH or mRNA for RNA FISH) in both eukaryotic and prokaryotic cells have been discovered. The methods described herein can be completed in less than 20 minutes in many instances. Moreover, the method allows for subsequent removal of probes thereby allowing for the same fixed cell to be repeatedly probed with different probes. This can be useful for example when the number of different distinguishable labels is limiting thereby limiting the number of different probes that can be used in methods where removal of probes is difficult or practically impossible.

Without intending to limit the invention it is believed that the methods described herein are so much faster than many other in situ methods at least in part because: 1) gentle fixation by crosslinking using chemical reagents such as formaldehyde and permeabilization by an organic solvent such as an alcohol which dissolves most lipids in cell membrane and nuclear membrane; 2) an extended heating step ensures thorough denaturation between double strands of genomic DNA (or RNA, including but not limited to, secondary structures or RNA-protein complexes for RNA FISH); 3) short oligonucleotide probes (e.g., with less than 150 nucleotides in length) allow for extremely fast diffusion through cellular and nuclear structures; and 4) a lack of crowding reagents such as dextran sulfate or blocking DNA reagents such as salmon sperm DNA and Cot-1 DNA typically used to prevent non-specific nuclear staining.

Any of a variety of cells or tissues can be used with the methods described herein. In general, it should be understood that “tissue” can comprise cells and thus “cells” can include cells that are in tissue or tissue samples. In some embodiments, the cells are prokaryotic cells such as bacteria or other microbes. In some embodiments, cells are a mix of eukaryotic cells and prokaryotic cells. In some embodiments, cells or tissues are eukaryotic cells only. In some embodiments, the eukaryotic cells or tissues are mammalian (e.g., human). Any tissue sample from a subject may be used. Examples of tissue samples that may be used include, but are not limited to, breast, prostate, ovary, colon, lung, endometrium, stomach, salivary gland or pancreas. The tissue sample can be obtained by a variety of procedures including, but not limited to surgical excision, aspiration or biopsy. The tissue may be fresh or frozen. In some embodiments, the tissue sample is fixed and embedded in paraffin or the like. If paraffin has been used as the embedding material, the tissue sections can be deparaffinized and rehydrated to water. The tissue sections may be deparaffinized by several conventional standard methodologies. For example, xylenes and a gradually descending series of alcohols may be used. Alternatively, commercially available deparaffinizing non-organic agents such as Hemo-De (CMS. Houston. Tex.) may be used.

Initially, cells are chemically fixed, e.g., on a microscope slide or other flat surface. Fixation of the cells will generally be for a short period of time (e.g., less than 10, 9, 8, 7, 6, or 5 minutes, e.g., 1-5 minutes). Fixation solution can include, for example, formaldehyde (e.g., 4%), which for example can be formed from paraformaldehyde in an aqueous solution. The fixation process can be ended by greatly diluting or otherwise removing the fixative, for example by successive washes. Wash solutions can be, for example, 1×PBS. Once the cells are fixed, they can be permeabilized further with a lipid-dissolving solvent. Exemplary solvents include but are not limited to acetone, alcohols such as methanol and ethanol. Permeabilization can occur for a short time, generally less than 10 minutes, for example 3-7 minutes, e.g., about 5 minutes. The permeabilization step can be ended with successive washes, which like above can optionally be 1×PBS washes to remove the solvent.

Cell fixation and permeabilization can happen simultaneously such as using formaldehyde or methanol only, or a mixed solution of acetic acid and methanol.

DNA (or RNA for RNA FISH) within the fixed and permeabilized cells can be denatured by contacting the cells with formamide and/or ethylene carbonate under heating conditions. For example the cells can be contacted with 50-90% (e.g., 60-90%, about 80%) formamide at 60-100° C. (e.g., 70-85 or about 80° C.). In some other embodiments, the cells can be contacted with (e.g., 15%) ethylene carbonate (e.g., for 10 minutes, and in some embodiments in higher temperatures to partially compensate for poorer performance of ethylene carbonate compared to formamide). In some embodiments, denaturation occurs for less than 20 minutes, e.g., between 5-15 minutes, e.g., for about 10 minutes.

A solid scaffold can be used to attach cells for in-situ hybridization. The scaffold can be optically transparent for microscopic imaging. The scaffold can also facilitate heat transfer efficiently. In some embodiments, the scaffold can be glass or polymer. In some embodiments, the scaffold is a 1 mm glass slide. In some embodiments, the scaffold is a #1.5 (˜0.17 mm in thickness) glass coverslip. In some embodiments, the scaffold is a #1.5 (˜0.17 mm in thickness) polymer coverslip. In some embodiments. #1.5 glass coverslips are chosen for rapid RNA FISH.

Permeabilized cells having denatured DNA (or RNA for RNA FISH) can subsequently be contacted with one or more oligonucleotide probes to introduce the probes into the nucleus of the cells for DNA FISH or into the cytoplasm for RNA FISH. In some embodiments, the oligonucleotides have less than 150 nucleotides in length. In some embodiments, the length of each oligonucleotides is less than 140 nucleotides (or alternatively less than 130, 120, 110, 100, 80, 70, 60, 50, 40, 30 nucleotides). In some embodiments, the cells are not contacted with dextran sulfate or blocking agents such as albumin, sperm DNA or other exogenous DNA (e.g., Cot-1 DNA) or in some embodiments, any of these agents. Thus, the oligonucleotide probe solution will not contain any components of at least 20%, 15%, 10%, 5%, 3%, 1%, 0.5% or 0.1% (w/v or w/w) that is more than 500,000 daltons (or alternatively more than 400,000, 200.000 Da, 100,000 Da, 80,000 Da, 50,000 Da, 30,000 Da, 10.000 Da, 8,000 Da, 5,000 Da, 3,000 Da. 1,000 Da).

The oligonucleotide probes (made, for example, of DNA, RNA, LNA, or any other kind of nucleotides) can be provided at a concentration such that signal will be detectable. For example, in some embodiments, the oligonucleotide probes are provided at from 10-500 nM, e.g., 50-200 nM. The choice of probe will depend on the genetic target of interest. In some embodiments, one or more genetic abnormality is of interest. Genetic abnormalities that can be detected by this method include, but are not limited to, amplification, translocation, deletion, addition and the like.

Probes can be labeled as desired, for example but not limited to with a fluorophore. Examples of fluorophores include, but are not limited to, rare earth chelates (europium chelates), Texas Red, rhodamine, fluorescein, dansyl, Lissamine, umbelliferone, phycocrytherin, phycocyanin., or commercially available fluorophors such SPECTRUM ORANGE™ and SPECTRUM GREEN™ and/or derivatives of any one or more of the above. Multiple probes used in the assay may be labeled with more the same label or with distinguishable fluorescent or pigment colors. These color differences provide a means to identify the hybridization positions of specific probes. Moreover, probes that are not separated spatially can be identified by a different color light or pigment resulting from mixing two other colors (e.g., light red+green=yellow) pigment (e.g., blue+yellow=green) or by using a filter set that passes only one color at a time. Probes can be labeled directly or indirectly with the fluorophore, utilizing conventional methodology. Alternatively, a non-fluorophore moiety can be used as a detectable moiety. Oligonucleotide probes can be modified before, during or after the FISH procedure to generate a detectable signal. For example, the probes can have an attached dye during the contacting step, a detectable functional group can be added post-staining (post-contacting), or another oligonucleotide sequence can be added (during or after the contacting of the probe to the target nucleic acid) that does not bind the target but can be subsequently hybridized to a dye-labeled “readout” single-strand DNA probe. Thus “labelled” can be understood to include having a physical structure that ultimately allows for detection of the oligonucleotide probes, either during or after contact of the probes to the target nucleic acid. Oligonucleotide probes can be conjugated with non-fluorescent labels such as biotin or added with a short single DNA strand to integrate with all kinds of signal amplification techniques such as tyramide signal amplification, branched DNA amplification, and hybridization chain reaction.

Various degrees of hybridization stringency can be employed. As the hybridization conditions become more stringent, a greater degree of complementarity is required between the probe and target to form and maintain a stable duplex. Stringency is increased by raising temperature, lowering salt concentration, or raising formamide concentration. Hybridization conditions can include for example, formamide, SSC, or other ingredients used in hybridization reactions.

Probes should have sufficient complementarity to the target nucleic acid sequence of interest so that stable and specific binding occurs between the target nucleic acid sequence and the probe. The degree of homology required for stable hybridization varies with the stringency of the hybridization medium and/or wash medium. In some embodiments, completely homologous probes are employed, but probes exhibiting lesser but sufficient homology can also be used.

Oligonucleotide probes will generally be DNA molecules but can also be RNA or can contain non-natural nucleotides. In some embodiments, the oligonucleotide are 13-55, or 13-40 or 30-70 or 30-100 nucleotides in length. In some embodiments, more than one probe is hybridized at the same time. For example, in some embodiments, 10-1000 oligonucleotides of different sequence are hybridized simultaneously. In some embodiments, the oligonucleotide(s) target repetitive DNA, in which case in some embodiments a single oligonucleotide sequence (albeit in multiple copies) can be used. The length of the nucleotides are chosen according to melting temperature and potential off-target binding sites in the genome. The length of oligonucleotides is related to stringency. In some embodiments, the longer the oligonucleotide, the more stringent hybridization and washing condition that can be used.

Following hybridization and before detection, one can perform one or more wash steps to remove unhybridized probe. For example, in some embodiments, lx PBS at room temperature (i.e., 23° C.) can be used to wash the cells. Depending on oligonucleotide length, in some embodiments, higher stringency washes (e.g., including 10-50% formamide) can be used at room or elevated temperature. In some embodiments, for example, the cells can be washed with 20-30% formamide at 20-37° C. depending on the oligonucleotide sequence and its length.

The probed samples may be analyzed by standard techniques of fluorescence microscopy (see for e.g. Ploem and Tanke, Introduction to Fluorescence Microscopy, New York, Oxford University Press (1987)). In some embodiments, each sample is observed using a microscope equipped with appropriate excitation filters, dichromic, and barrier filters. Filters are chosen based on the excitation and emission spectra of the fluorochromes used. Photographs of the slides may be taken with the length of time of film exposure depending on the fluorescent label used, the signal intensity and the filter chosen. Exemplary detection systems are described in, e.g., U.S. Pat. No. 8,542,899 (Automatic image analysis and quantification for fluorescence in situ hybridization) and 8,000,509 (Image processing method for a microscope system).

In order to correlate cellular morphology with probe signals, one may use a computer-driven, motorized stage which stores location of co-ordinates. This may be used to evaluate the same area by two different analytical techniques. For example, color images of morphologically stained areas may be captured and saved using a computer-assisted CCD camera. The same section may be subsequently taken through the procedure described herein, the stored locations recalled, and the designated areas scored for the presence of fluorescent nuclear signals. This can also be done for different probings of the same tissue or cell sample. Alternatively, DAPI staining of nuclei can be used as a marker for image registration.

Hundreds of cells can be scanned in a tissue sample and quantification of the specific target nucleic acid sequence can be determined in the form of fluorescent spots, which are counted relative to the number of cells. Deviation of the number of spots in a cell from a norm may be indicative of a malignancy or a predisposition to a malignancy, disease, or other abnormality. The relative number of abnormal cells to the total cell sample population may also indicative of the extent of the condition or abnormality. In addition, using family health histories and/or genetic screening, it is possible to estimate the probability that a particular subject has for developing certain types of cancer or genetic diseases. Those subjects that have been identified as being predisposed to developing a particular form of cancer can be monitored or screened to detect early evidence of disease.

One advantage of the in situ methods described herein is that following detection a first probe set can be removed from the cells or tissue and one or more further oligonucleotide probe sets can be used to probe the same cells or tissue. Probes can be removed from the cells or tissue with any number of washes. An exemplary wash is can include formamide at room temperature or elevated temperature to strip the oligo nucleotide probes from the cells. For example, 80% formamide at 50 C can be used to wash the cells for 30 seconds to remove bound oligonucleotide probes.

Having washed away the probes, the method can be repeated. As the cells are already fixed from the first round, a fixation step need not be repeated. Thus, the second and subsequent rounds of probing with different probe sets can begin by introduction of the new probes into the cells, washing away unbound probe, and detection of binding of the new probe set.

### EXAMPLES

### Example 1

The following examples are offered to illustrate, but not to limit the claimed invention.

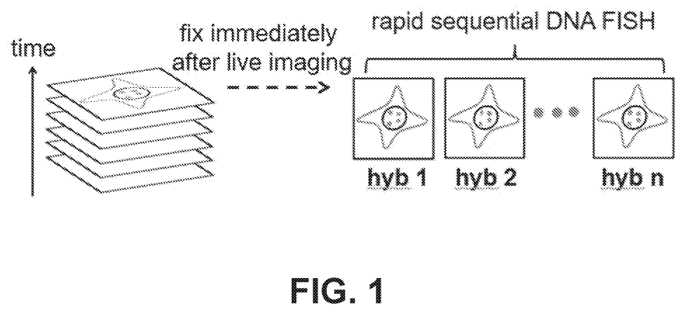

Here we report a correlative imaging method that combines the dynamic tracking capability of CRISPR imaging with the multiplicity of sequential FISH. This method allows us to perform live-cell CRISPR imaging first to obtain the dynamics of many genomic loci using one Cas9 protein and the corresponding sgRNAs followed by sequential rounds of DNA FISH to decode loci identity (FIG. 1).

### Materials and Methods

### Cell Culture

Human retinal pigment epithelium (RPE) cells (ATCC, CRL-4000) were maintained in Dulbecco's modified Eagle medium/Nutrient Mixture F-12 (DMEM/F-12) with GlutaMAX supplement (Gibco) in 10% Tet-system-approved fetal bovine serum (FBS) from Clontech. Human embryonic kidney (HEK) cell line HEK293T were maintained in DMEM with high glucose (UCSF Cell Culture Facility) in 10% Tet-system-approved FBS (Clontech). Cells were maintained at 37° C. and 5% CO2 in a humidified incubator.

Lentiviral Production and Stable Expression of dCas9, sgRNA, and Fucci Constructs

For viral production, HEK293T cells were seeded onto 6-well plate 1 day prior to transfection. 0.1 μg of pMD2.G plasmid, 0.8 μg of pCMV-dR8.91, and 1 μg of the lentiviral vector (Tet-on 3G, dCas9-EGFP, sgRNA, or Fucci) were cotransfected into HEK293T cells using FuGENE (Promega) following the manufacturer's recommended protocol. Virus was harvested 48 hr post transfection. For viral transduction, cells were incubated with culture-medium-diluted viral supernatant for 12 hr. RPE cell lines stably expressing dCas9-EGFP were generated by coinfecting cells with a lentiviral cocktail containing viruses encoding both dCas9-EGFP and the Tet-on 3G transactivator protein (Clontech). Clonal cell lines expressing dCas9-EGFP were generated by picking a single-cell colony. The clones with low basal level expression of dCas9-EGFP were selected for CRISPR imaging. Clonal RPE cell line expressing dCas9-EGFP were transduced with lentivirus encoding Fucci (Gemini::RFP and Cdt1::mIFP linked by P2A domain) and cells with stable expression of Fucci was sorted using flow cytometry. The Fucci-containing cell line showed normal cell division and cell cycle progression.

### Optical Setup and Image Acquisition

Fluorescence images were acquired on an inverted wide-field microscope (Nikon, Ti-E) with a 100×1.45 N.A. oil immersion objective. The custom-build epi-illumination optics (Lumen Dynamics, X-Cite XLEDI) provided excitation in DAPI, FITC, Cy3, and Cy5 channels. Quad-band dichroic excitation filter (Semrock, ZT405/488/561/640) was installed in the excitation path and quad-band emission filter (Semrock, FF410/504/5821669) in the emission path. Additional emission filters at 525 nm and 595 nm with 50 nm bandwidth were used for emission in FITC and Cy3 channels respectively to further reduce background noise. They were mounted into a motorized filter-wheel (Sutter Instrument, Lambda 10-B). A motorized microscope stage (ASI) controls the xy and z translation of the sample. The images were recorded with a sCMOS camera (Hamamatsu, C11440) in z-stacks of 6 μm with 0.3 μm steps. The microscope, light source, motorized stage, motorized filter wheel, and camera were controlled through custom configuration in Micro-Manager software. Live imaging was performed in FITC channel and FISH imaging was performed in Cy5 channel unless noted otherwise. Each image in Cy5 channel has a corresponding image in DAPI channel at the exactly same position to use in the two-step image registration algorithm described later. To improve mIFP signal, a final concentration of 25 μM biliverdin (Sigma 30891) was added to Fucci-containing cells at 12 hr before live imaging in FITC, Cy3, and Cy5 channels.

### DNA Fish

The bottom coverglass surface of an 8-well imaging chamber (Nunc Lab-Tek II. Thermo Fisher) was coated with 0.01% poly-L-lysine solution (Sigma) solution for 15 minutes and rinsed with PBS buffer three times. Cells were allowed to attach to the #1.5 coverglass (˜0.17 mm in thickness) surface for overnight. Cells were fixed with 4% paraformaldehyde solution (Chem Cruz) at room temperature for 5 min followed by PBS buffer wash for three times. Cell membrane and nucleus membrane were permeabilized by methanol incubation for 5 min followed by PBS buffer wash. Cells were then heated on a hot plate at 80° C. for 10 min in 80% formamide (Sigma). Cells were incubated for 2 min in hybridization solution of 200 nM oligo probes in the presence of 50% formamide and 2×SSC followed by PBS buffer wash three times. The conventional hybridization reagents such as dextran sulfate and blocking DNA reagents were not required. Imaging was performed in imaging buffer containing glucose, glucose oxidase, and catalase to prevent photobleaching. After each round of imaging in sequential FISH, the cells were washed with 80% formamide at 50° C. for 30 s to remove the bound oligo probes followed by a new round of probe hybridization.

### Two-Step Image Registration Algorithm

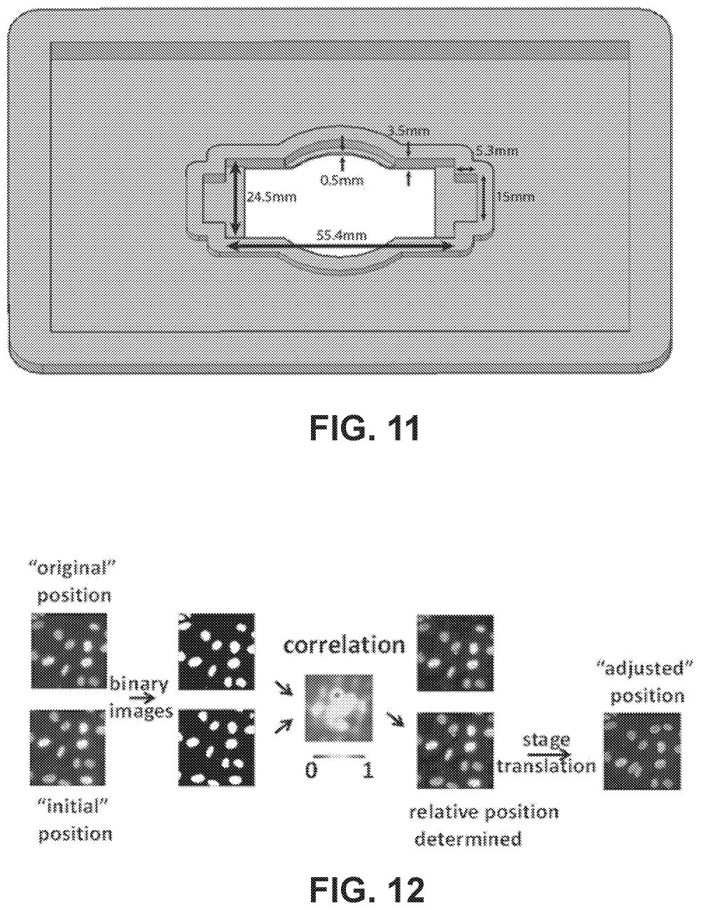

To improve the reproducibility of sample positioning during repetitive mounting and unmounting steps, we designed a 3D-printed stage adaptor (FIG. 11l) that ensures tight fit of 8-well chambers on microscope stage. To precisely registered the acquired live and fixed images, we first performed a stage registration step (FIG. 12 in the Supporting Material) that allows the original region to be found after the sample is put back onto microscope stage. The position of the sample cell in live imaging at the last frame is termed as “original.” The position of the sample cell immediately after it is put back on stage is considered as “initial.” Image of nuclei at the initial position was convoluted to the image of nuclei in the original position to calculate full-image correlation. The images were down-sampled (a fraction of pixels, for example, 1/10, in both x and y) to speed up the registration algorithm for near real-time feedback in sample positioning. The images were then converted to binary format and convoluted to calculate correlation. The peak position in correlation corresponded to the original position in live imaging and the compensatory stage displacement to return to the original position was calculated based on the pixel size. The algorithm takes only a few seconds to process two 1024×1024 images. The motorized stage was then translated to the “adjusted” position based on the input of translation displacement. The spatial precision of this algorithm depends on the down-sampling and is usually found within 1 μm, consistent with the precision expectation at this step because the distance between every 10 pixels in the x or y direction is about 1 μm. Typically, the “initial” position is within the field of view of the “original” position. However, if necessary, a much larger imaging region, on millimeter scale, could be rapidly scanned and tiled using Micro-Manager and ImageJ stitching plugin to find the “original” position. The algorithm works well based on similar features between images. For example, when nuclei shape can be acquired in live imaging (e.g. diffusive nuclear EGFP signal in the current system), the correlation could be done between the last frame of live imaging and fixed DAPI signal. Alternatively, on stage DAPI staining is performed before the sample is taken off stage. The algorithm works well for both a single slice and a z-stack of nuclei.

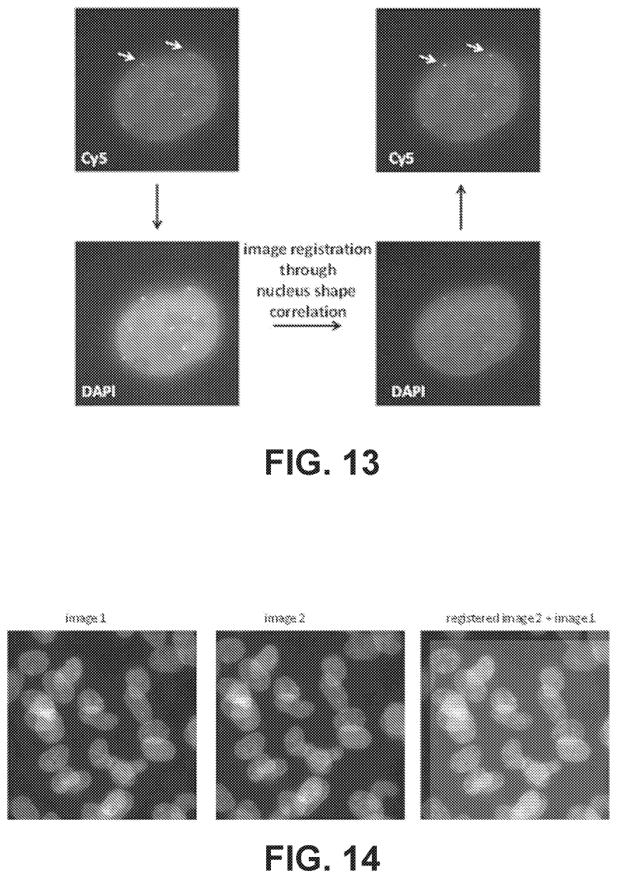

Second, a further refinement in image registration was applied in image analysis to register between CRISPR loci at the last frame of live-cell imaging and FISH spots (FIG. 13 in the Supporting Material). Based on the nuclei shape, images taken in live EGFP channels at the last frame and fixed DAPI channels were registered and then the same image registration operation was applied to overlay the last frame of live images and FISH images. The registration algorithm is a modified version of image registration (Guizar-Sicairos, M., S. T. Thurman, and J. R. Fienup. 2008. Opt Lett 33:156-158) that accounts for sample rotation and achieves subpixel precision through up-sampled discrete Fourier transforms (DFT) cross correlation. Briefly, the algorithm rotates an image in 1-degree steps and estimates the two-dimensional translational shift to register with a reference image through calculating the cross-correlation peak by fast Fourier transforming (FFT). The highest peak corresponds to not only the two-dimensional translational shift estimate but also the optimal angle of rotation. A refined translational registration with subpixel precision is then achieved through up-sampling the DFT in the small neighborhood of that earlier estimate by means of a matrix-multiply DFT. Our algorithm registered the images to a precision within 1/10 of a pixel. As the algorithm operates on Fourier Transform in frequency domain, it remains robust in image registration when features are more densely distributed (FIG. 14 in the Supporting Material).

### Single Particle Tracking

The positions of the spots in cell nuclei in live images were determined in CellProfiler. The position information at different time points is linked to generate trajectories using custom-written MATLAB (The MathWorks, Natick, Mass.) codes.

### Measurement of Loci Intensity

Z-stack images were first projected to generate an image using maximum-z projection and intensity measurement is performed on the projected images using custom-written MATLAB codes. The peak intensity of the genomic loci puncta was measured as the peak value in the selected region of interest subtracting nuclear background. The nuclear background was calculated as the mean value in nucleus regions lacking detectable puncta.

### Target Genomic Loci

We use hg19 version of human genome. The regions involved in this study are Chr1: 2581275-2634211; Chr3: 195505721-195515533 (denoted as Chr 3); Chr3: 195199025-195233876 (denoted as Chr3*); Chr7: 158122661-158135328; Chr13: 112930813-112973591 Chr19: 44720001-44760001 (denoted as NR1); Chr19: 29120001-29160001 (denoted as NR2); ChrX: 30806671-30824818.

sgRNA protospacer sequence to image human
genomic loci
sgChr3, 5′ GUGGCGUGACCUGUGGAUGCUG 3′
sgChr7, 5 GCUCUUAUGGUGAGAGUGU 3′
sgChr13, 5′ GAAGGAAUGGUCCAUGGUUACC 3′
sgChrX, 5′ GGCAAGGCAAGGCAAGGCACA 3′
DNA FISH probe sequence to image human
genomic loci
Chr1, 5′ CCAGGTGAGCATCTGACAGCC 3′
Chr3, 5′ CTTCCTGTCACCGAC 3′
Chr3*, 5′ CCACTGTGATATCATACAGAGG 3′
Chr7, 5′ CCCACACTCTCACCATAAGAGC 3′
Chr13, 5′ GGTAAGCATGGACCATTCCTTC 3′
ChrX, 5′ TTGCCTTGTGCCTTGCCTTGC 3′
Telomere, 5′ CCCTAACCCTAACCCTAA 3′
Centromere, 5′ ATTCGTTGGAAACGGGA 3′

### Non-Repetitive Probe Design and Synthesis

Oligo pool library was designed such that seven modules were concatenated. Two sets of index primer pairs were used to amplify the entire oligo pool library or selectively a sub-library of oligos. A variable region was designed to cover a genomic region of interest. Typically, 200 probes tiling over 40-kb genomic region lead to detectable FISH signal. The 30-nucleotide variable region was flanked on one side by T7 promoter used in in-vitro transcription and on the other side by a reverse transcription primer sequence shared in the entire library. The sequence in the variable region was first designed in OligoArray 2.1 software using parameter set −n 20−1 30 −L 30 −D 1000 −t 70 −T 90 −s 76 −x 72 −p 35 −P 80 −m “GGGG;CCCC;TTTTT;AAAAA”. Sequences with homology of 17 nucleotides or more to the human genome were detected with blast+ and removed. Sequence with homology of 14 nucleotides or more due to concatenation between variable region and reverse transcription primer were also removed. The index primers and reverse transcription primers were designed by first truncating sequences to 20-mer oligo library from a 25-mer random oligo library (Xu, Q. K. 2009. Proc Natl Acad Sci USA 106:2289-2294). The oligos with a melting temperature between 75° C. and 83° C. were selected. Sequences with homology of 11 nucleotides or more or 5 nucleotides or more homology to the 3′ end within the 20-mer oligo library were removed. Oligos without a G or C base in the last two nucleotides on the 3′ end were removed. Sequences with homology to T7 promoter sequence were removed. Non-repetitive probe sequence files for loci NR1 and NR2 are included in the Supporting Material.

The oligo pool library is synthesized by CustomArray Inc and amplified by limited cycle PCR. In-vitro transcription (NEB, E2040S) was performed and dsDNA was converted to RNA with an effective amplification of 200+fold. The RNA was then converted back to ssDNA in reverse transcription reaction (ThermoFisher, EP0751). The final amplified DNA oligonucleotide library comprised a pool of oligos with less than 70 nucleotides in length.

### Results

### Fast FISH Staining of Genomic DNA

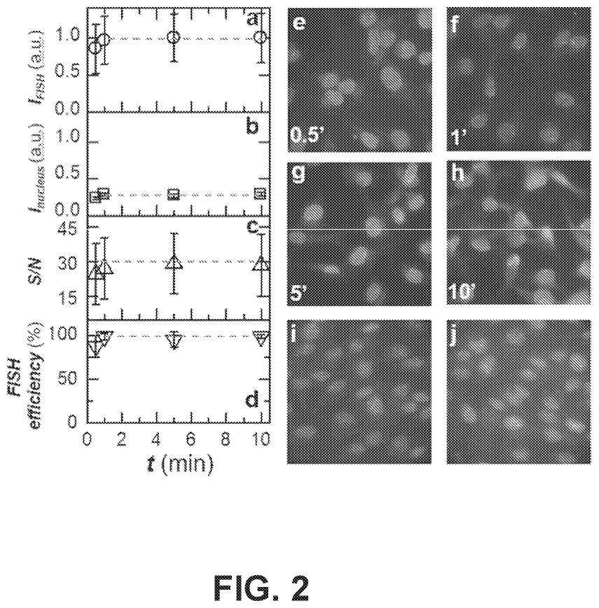

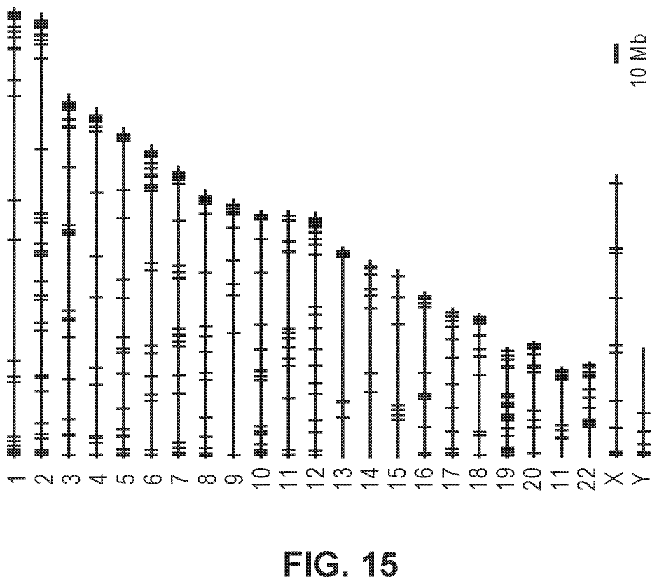

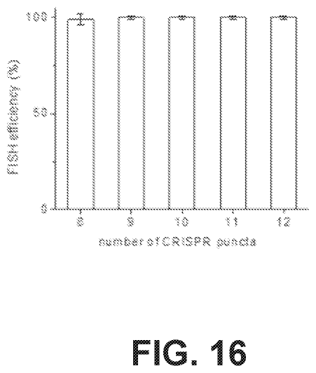

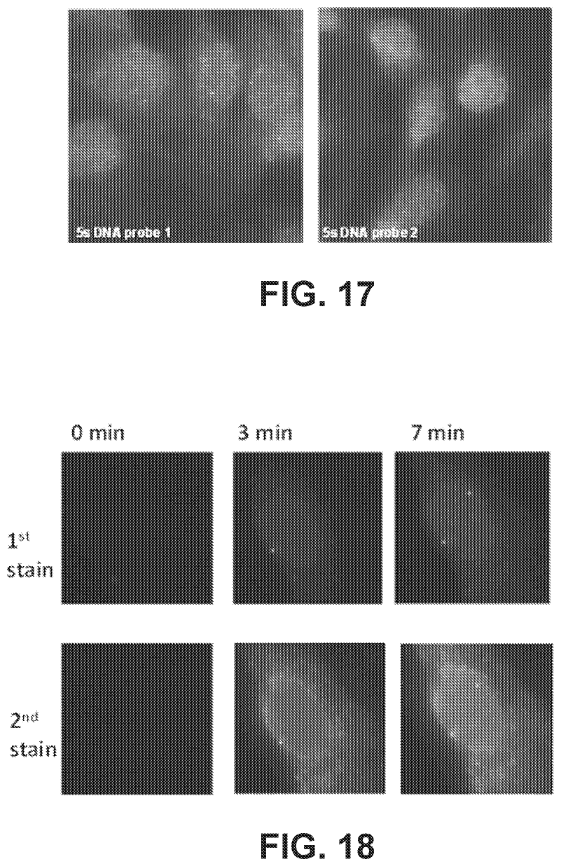

In order to practically and efficiently perform multiple rounds of DNA FISH on the same sample, we sought to address the technical challenge that DNA FISH often requires many hours or overnight for probe hybridization, which is much longer compared to that needed for RNA FISH (2-4 hours) because of the double-stranded nature of DNA. We speculate that a rapid binding of oligo probes directly to genomic DNA is possible after sufficient DNA denaturation to separate duplex strands. To simplify the initial test, we started with oligo DNA probes targeting the tandemly repetitive sequence (TRS) in the human genome (FIG. 15 in the Supporting Material) so only one FISH probe is needed (Benson, G. 1999. Nucleic Acids Res 27:573-580). FIG. 2a-d shows the kinetics of FISH staining targeting a TRS region. Under optimized conditions, staining is essentially complete in 1 min and acceptable signal-to-noise ratio is even achieved in as short as 0.5 min, in drastic contrast to the common practice of overnight incubation. The representative images of cells stained for different durations show that the nuclear background is consistently low throughout staining. The efficiency of FISH staining, calculated as the ratio of observed to expected number of FISH puncta, reaches almost 100% by 1 min (FIG. 2d; FIG. 16 in the Supporting Material). The signal-to-noise ratio reaches ˜30 fold for the ˜800 copies and we thus estimate a lower detection limit to be ˜40 probes (FIG. 17 in the Supporting Material), a number consistent with those reported in recent RNA FISH studies. The signal-to-noise, nuclear background, and FISH efficiency remain constant after 1 min so a wide time window of hybridization works well. To test whether the optimized FISH protocol could also be widely applied to label non-repetitive genomic sequences, we designed oligo DNA probe pools that tile non-repetitive genomic regions (Beliveau, B. J. 2012. Proc Natl Acad Sci USA 109:21301-21306) and observed similarly efficient staining of these regions (FIG. 2i-j). Typically, we observe two puncta in each cell nucleus as expected for the diploid cells. Thus, this method can rapidly detect aneuploidy in interphase cells and potentially report copy number variations with probes targeting specific genes of interest.

Three aspects could have contributed to the fast staining: 1) gentle fixation by crosslinking and permeabilization by an alcohol wash dissolves most lipids in cell membrane and nuclear membrane; 2) an extended heating step ensures thorough denaturation between double strands of genomic DNA—we find that the genome accessibility directly correlates to the heating denaturation (FIG. 18 in the Supporting Material); and 3) short oligo DNA allows extremely fast diffusion through cellular and nuclear structures. Note that the current FISH protocol is also greatly simplified in procedure, requiring no crowding reagents such as dextran sulfate to boost probe diffusion or blocking DNA reagents such as salmon sperm DNA and Cot-1 DNA to prevent non-specific nuclear staining.

### Multiple Sequential Rounds of DNA FISH in Multiplex Imaging

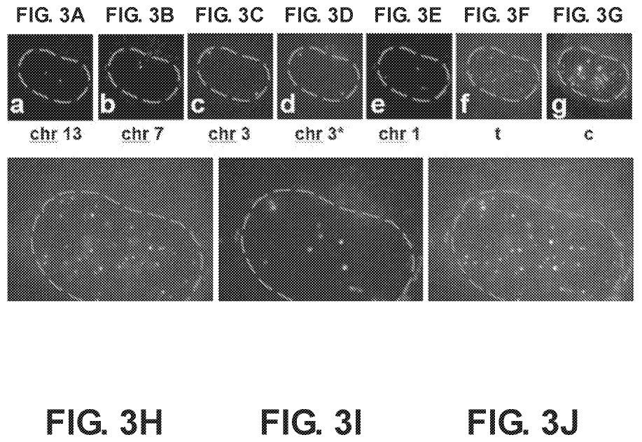

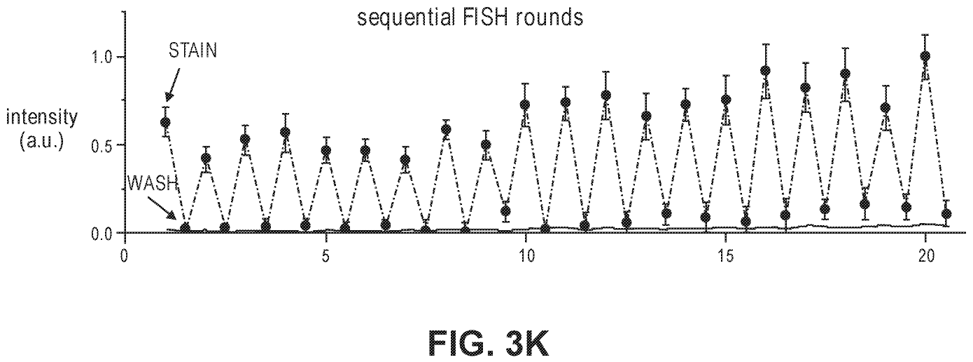

With the ease of performing rapid FISH staining, we then tested whether this technique can be applied to multiple sequential rounds of hybridization. A technical challenge here is to minimize the interference of bound probes with the next round of imaging. In previous studies, this issue was addressed by either DNase enzymatic reactions to degrade DNA probes bound to RNA targets (Sakaue-Sawano. A. 2008. Cell 132:487-498) or photobleaching the dyes on bound probes using powerful lasers and then adding new probes targeting other vacant binding sites (Chen, K. H. 2015. Science 348:aaa6090). Here to explore a simpler protocol, we apply a stringent wash step using concentrated formamide solution at elevated temperature to strip the bound oligo DNA probes after each round of imaging and detection. FIG. 3 a-g shows an example of sequential FISH rounds where five distinct genomic regions, telomere, and centromere are sequentially detected through six rounds of staining and washing. The images show effective staining of specific sequences without interfering loci signals between sequential rounds. We find that the wash step is efficient and probes are effectively removed within 0.5 min. In FIG. 3, we have also demonstrated two color FISH (Cy3 and Cy5 channel) to distinguish two loci as close as 300 kb away on chromosome 3 in one FISH round, suggesting the multiplex imaging capacity of the method could be further expanded through combining multi-color approaches.

Recent studies show that multiple rounds can quickly expand the multiplex capacity to thousands or more through various encoding strategies (Lubeck, E. 2014. Nat Methods 11:360-361; Chen, K. H. 2015. Science 348:aaa6090; Wang, S. 2016. Science 353:598-602). To further test the potential of the method for multiplex imaging, we measured intensities of a specific genomic region through 20 rounds of alternating staining and washing. FIG. 3k shows that the loci can be reversibly and consistently stained for at least 20 rounds without visible sample deterioration. High contrast is reproducibly seen with consistently high intensity after staining and almost negligible signal after wash. In fact, the residual signal after washing is so dim that it is often undetectable from nuclear background, especially in earlier rounds. The consistent and reversible staining and washing in multiple DNA FISH rounds suggest the multiplex potential of the method.

Sequential DNA FISH after Live Imaging Resolves Loci Identity

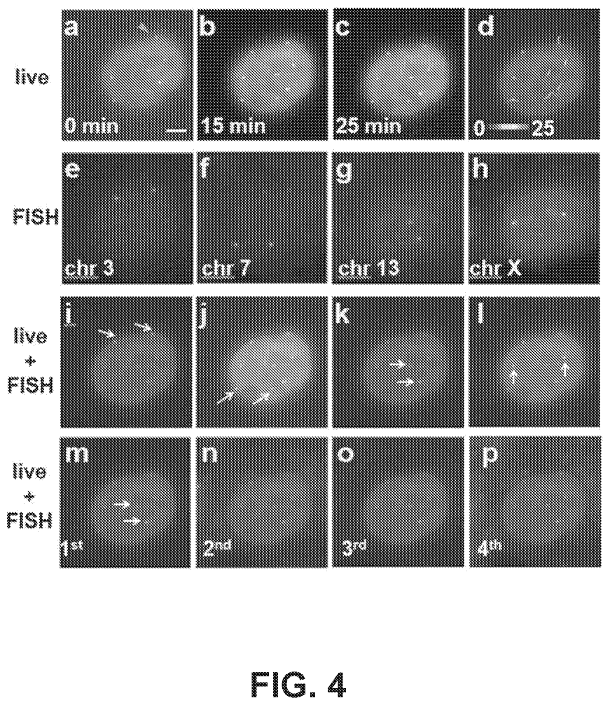

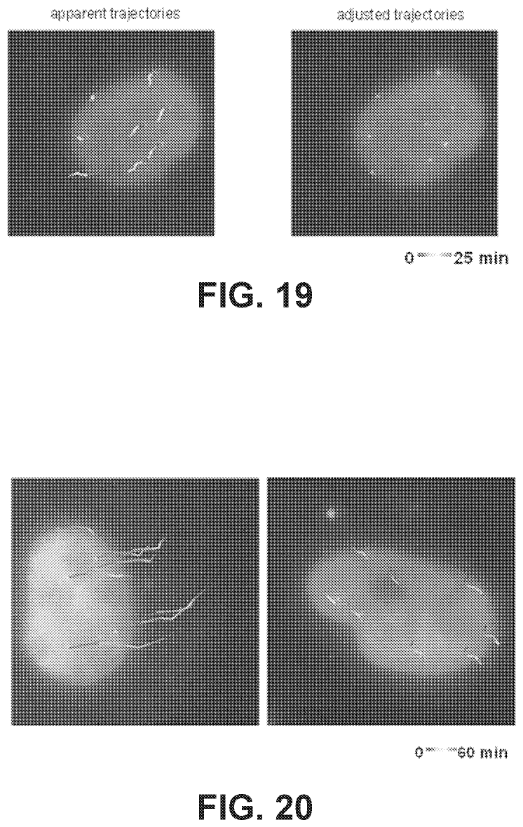

FIG. 4a-d shows representative time-lapse images of a cell with simultaneous labeling of multiple genomic loci. Here, four sgRNAs with different protospacer sequence are expressed in the cell nucleus at the presence of dCas9::EGFP fusion protein, resulting in efficient labeling of the corresponding genomic loci and eight bright spots in the nucleus as homologous chromosomes are simultaneously labeled in the diploid cell. As these sgRNAs share the same dCas9 binding motif, one cannot directly distinguish the loci identities based on the live images. Nonetheless, genome dynamics can be extracted regardless of the loci identities. Comparison of images at different time points (FIG. 4a-c) suggests that the relative positions of these loci are essentially stable, consistent with earlier FRAP measurement on cell nuclei (Strickfaden, H. et al. 2010. Nucleus 1:284-297; Gerlich, D. et al. 2003. Cell 112:751-764). The trajectory over 25 min overlaid on live images reveals a global motion of the nucleus (FIG. 4d). The adjusted chromosome dynamics after subtracting the contribution of global nucleus movement show a more randomized trajectory direction (FIG. 19 in the Supporting Material). Furthermore, we show in FIG. 20 that the global movement of nucleus is quite commonly seen, more so on longer observation timescale. We find that this global scale of nucleus movement is not caused by stage drift as cells in the same field of view have randomized trajectory direction with respect to one another. The live images also capture the dynamic vibrations between sister chromatids (arrowheads in FIG. 4a), which are sometimes distinguishable when the distance exceeds the optical diffraction limit.

The cells are fixed at the end of the live observation and prepared for sequential DNA FISH. To correlate live imaging of genomic loci with sequential FISH, it is desirable to register the same area with sub-micrometer precision to correlate images between live condition and fixed FISH rounds. This task is challenging, especially since the denaturation of DNA duplex strands during DNA FISH preparation requires elevated temperatures; therefore, the sample has to be taken off the microscope stage. To address this issue, we use a combination of three measures to achieve faithful image registration. First, a 3D printed microscope stage adaptor ensures consistent sample orientation by restricting sample rotation (FIG. 11 in the Supporting Material). Second, we employ a rapid correlation-based algorithm to find the same cell observed in live imaging. Finally, a more sophisticated registration algorithm (Guizar-Sicairos, M., S. T. Thurman, and J. R. Fienup. 2008. Opt Lett 33:156-158) that accounts for both translation and rotation is applied to register the live and fixed images based on the nucleus shape.

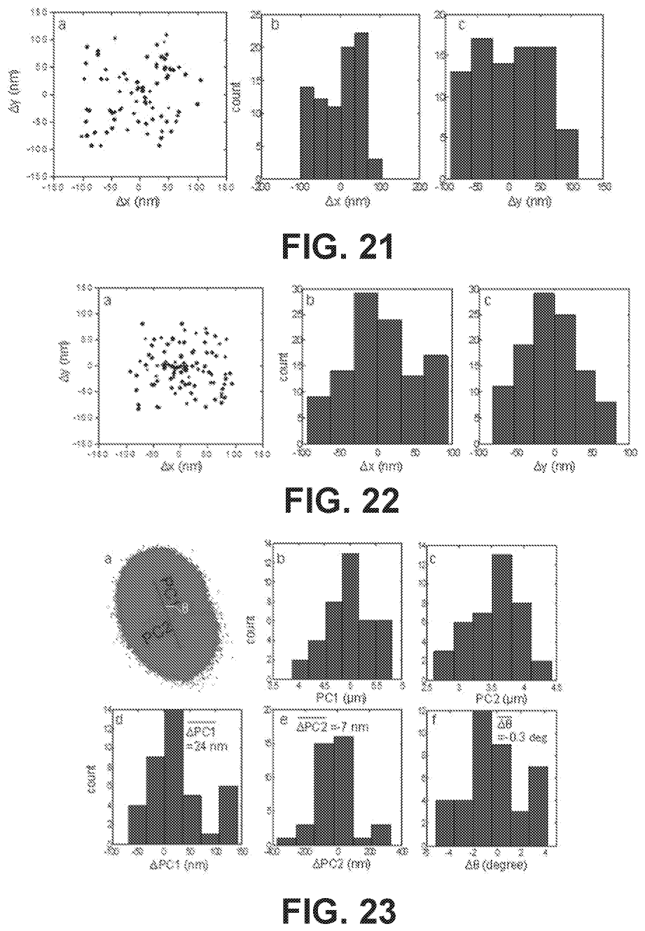

The identities of the four genomic loci are resolved in four rounds of DNA FISH as probes specific to each locus are sequentially introduced in each round (FIGS. 4e-h). Furthermore, overlay of the live-cell images and fixed-cell images demonstrate consistent loci position between live condition and fixed condition across various loci with a root mean square (RMS) error of ˜52 nm in loci registration (FIG. 4i-1; FIG. 21 in the Supporting Material). Similarly, we find images between sequential FISH rounds for a given probe register well with an RMS error of ˜43 nm (FIG. 4m-p; FIG. 22 in the Supporting Material). The successful image registration and principle component analysis on nuclear morphology (FIG. 23 in the Supporting Material) also confirms that negligible deformation is introduced during the FISH preparation steps. A closer inspection of FIG. 4m-p shows that positions of sister chromatids and the distances between them are faithfully maintained in repeated sequential rounds and registered well with the live image, further confirming the method maintains almost intact nuclear morphology at both global and local scale and is compatible with high-resolution imaging to resolve fine chromatin structure.

### Multi-Color Live Imaging Enabled by Correlative CRISPR Imaging and Sequential DNA FISH

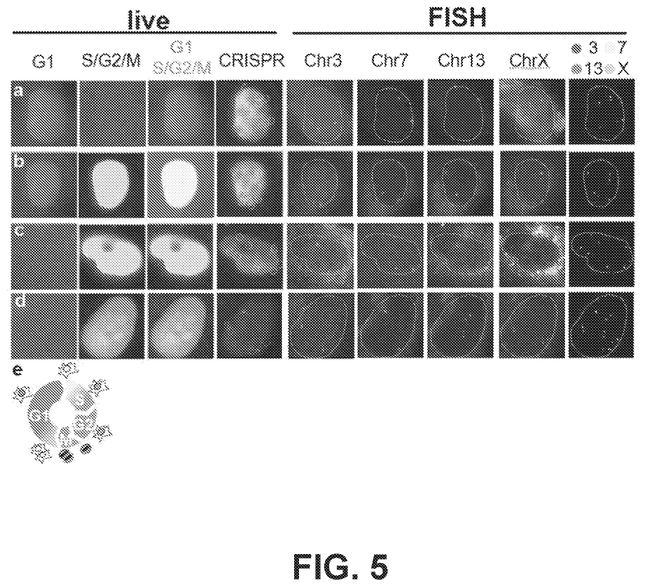

Because the correlative CRISPR imaging and sequential DNA FISH uses a single color channel in live imaging to track multiple genomic regions, it opens up other color imaging channels in live cell imaging to extract information otherwise difficult to obtain. Here we demonstrated this capability by performing CRISPR imaging of 4 loci in cells expressing the Fucci probe, a widely used cell-cycle tracker which uses two colors to mark G1 or S/G2/M cell phase respectively (Sakaue-Sawano. A. 2008. Cell 132:487-498). With the help from Fucci probe, we were able to distinguish G1 phase cells and early S phase cells (FIG. 5a-c), both of which display singlet spots for each locus in CRISPR imaging results, while late S/G2 phase cells could identified by doublet spots corresponding to replicated sister chromatids (FIG. 5d). Because the Fucci reporter itself already occupies two of the three color channels (green, orange and far red) that multi-color live cell imaging can be easily performed, this experiment is challenging for other multi-color CRISPR imaging methods. Potentially, with Fucci reporting the onset of S phase and CRISPR image detecting the replication of given genomic loci (e.g. based on intensity change of the labeled spot), we can profile replication timing and test how it affects the spatial organization of genome and the formation of topologically associated domains (TADs) formation (as recently proposed based on Hi-C results (Pope, B. D. 2014. Nature 515:402-405)). In addition, with other color channels opened up, our approach can also to be used to monitor the interaction of genomic loci with other nuclear components such as lamin, nuclear pore complex, and various nuclear bodies (Wang, Q. 2016. Nat Commun 7:10966), many of which are known to be active genome organizers.

### Discussion

The current method thus combines the advantage of acquiring dynamics in live-cell imaging and multiplex imaging capacity in sequential FISH. It frees up other in vivo color channels for imaging applications such as RNA expression and processing (Masui, O et al. 2011. Cell 145:447-458; Levesque, M. J., and A. Raj. 2013. Nat Methods 10:246-248), protein expression (Clowney, E. J. 2012. Cell 151:724-737; Wood, A. M. 2011. Molecular Cell 44:29-38) and various nuclear components which are closely related to the spatial organization and dynamics of the genome. Moreover, a simpler system of live imaging with more uniform expression and assembly of Cas9 protein and sgRNA could potentially reduce system variability and facilitate further quantitative analysis. As DNA FISH has demonstrated the power to probe genome organization in high-throughput fashion such as HIP-map (Shachar, S. 2015. Cell 162:911-923) and with super-resolution imaging using Oligopaints (Beliveau, B. J. 2015. Nat Commun 6:7147; Boettiger, A. N. 2016. Nature 529:418-422), the current work adds a new dimension of dynamic information. As the method of image registration between live and fixed conditions is directly transferrable to other systems, the concept of multiplex imaging through correlative imaging between live and fixed cells could be similarly applied to RNA imaging resolved by sequential RNA-FISH and protein imaging by sequential antibody staining.

### Conclusion

In summary, we introduce a correlative imaging method that combines the dynamic tracking capability of CRISPR imaging and the multiplicity of sequential FISH. After live imaging to obtain dynamics information of multiple genomic loci using one-color CRISPR system of one Cas9 protein and multiple sgRNAs, we perform rapid sequential rounds of DNA FISH to resolve loci identities. We also demonstrate a greatly simplified DNA FISH protocol that effectively stains genomic DNA in as short as 30 s in contrast to the common practice of overnight incubation. Our correlation-based algorithm to faithfully register between live images and fixed images can be readily adapted for other multiplex imaging applications.

### Example 2

A rapid and sequential RNA FISH method was performed as follows:

1) RPE cells were plated on polylysine-coated coverslip and the cell were allowed to attach overnight.

2) The cells were treated with ice-cold methanol for 4 min, and then 4% paraformaldehyde (PFA) for 5 min (the order of MeOH and PFA can be reversed). Alternatively, cells were treated with 4% formaldehyde at room temperature only for 5-15 min.

3) The cells were submitted to an 80° C. denaturation in 80% formamide for 10 minutes.

4) GAPDH probes and Neat1 probes were added and incubated for a 3-5 min hybridization (probe concentration was ˜120 nM) at 37° C. The hybridization solution was 2×SSC (0.3 M NaCl and 0.03 M sodium citrate, at pH 7.0), 10% formamide, without dextran sulfate.

5) The cells were then washed for 1-2 min at 37° C. The wash solution was 2×SSC, 10% formamide.

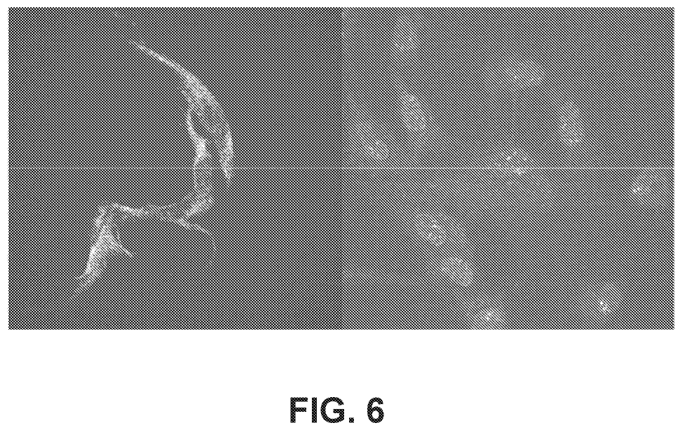

6) Finally the cells were imaged in 2×SSC. An example image is shown in FIG. 6.

One notable difference between the above protocol and existing RNA FISH protocols is that the protocol contains a heat denaturation step, similar to the heat step described herein for DNA. While the method can be performed without a heat denaturation step, the time required for the method is greatly shortened by including a heat denaturation step.

The above protocol was repeated with a series of different concentration (from 5% to 20%, w/v) of dextran sulfate with different molecular weight (5000 Da, 8000 Da, 15,000 Da, 40,000 Da and >500,000 Da) during probe hybridization in RPE and HeLa cells (stage 4). While the presence of dextran sulfate with low molecular weight (<=10,000 Da) did not have any positive or negative effect at short time scale involved. The presence of <10% of dextran sulfate with high molecular weight (>10,000 Da) didn't affect the hybridization speed significantly as well. The presence of high concentration (10%-20%) of dextran sulfate with high molecular weight (>10,000 Da) accelerated the hybridization speed but also increased non-specific binding when heat denaturation was not sufficient to denature all RNAs. Using higher temperature and/or longer duration of heating time, RNA could be fully denatured. Therefore, high concentration of dextran sulfate with high molecular weight, which is used by conventional DNA FISH, is not necessary for rapid RNA FISH here.

For rapid and sequential RNA FISH, once the first round of oligo probes was done, the same method developed in Example 1 for rapid and sequential DNA FISH could be used here. Typically, incubating the sample in a 2×SSC buffer with 50% formamide at 50° C. for 1-2 min, >99% oligo probes for detecting RNA were removed efficiently. For better stringency of probe stripping, higher temperature and higher concentration of formamide, multiple rounds of wash could be used when necessary. After probe stripping by heated washing buffer, a new set of oligo probes were hybridized with the sample at 37° C. for 3-10 min to detect the RNAs in a new round of RNA FISH. In this way, the same hybridization and probe stripping steps above were iterated for multiple rounds of rapid and sequential RNA FISH.

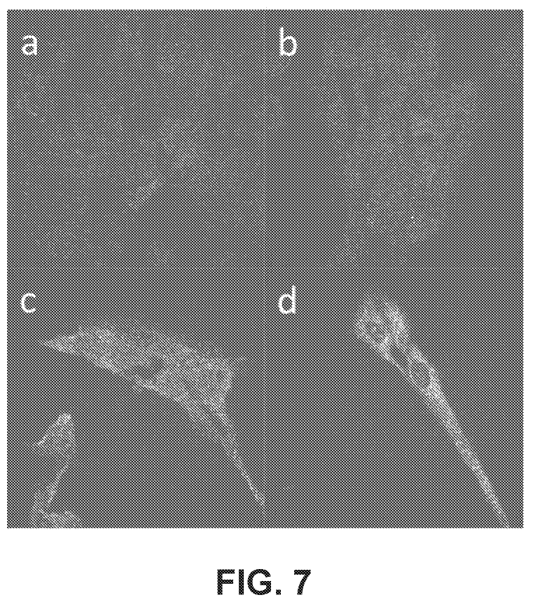

The effect of heat denaturation at different temperature for RNA FISH was further evaluated. A series of different temperature (37° C., 50° C., 70° C. and 80° C.) were tested to evaluate how much different temperature affect the hybridization speed. The denaturation buffer used here was 80% formamide and 2×SSC. The hybridization buffer used here was 10% formamide and 2×SSC. The oligo probes for human RNA GAPDH were used here. The concentration of oligo probes in all experiments done here were 100 nM. Higher concentration of probes could be used but not necessary. As shown from FIG. 7a to 7d, with the increase of heating temperature, stronger and denser binding signal was observed in RPE cells, suggesting heat denaturation promoted oligo hybridization with RNA targets in cells efficiently and significantly. By measuring the intensity of individual pixels, the fluorescent spots with the top 10% highest intensity in each image increased their intensity by 50% to 200% with every 10° C. of temperature rise.

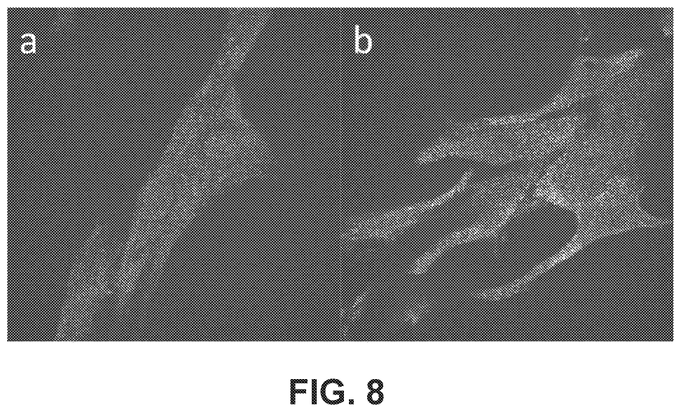

Longer hybridization time at 37° C. without heat denaturation but a simple hybridization buffer developed here (10% formamide and 2×SSC) were also developed here. In contrast to other well-known RNA FISH methods such as Stellaris RNA FISH sold by LGC Biosearch (requiring at least 4 hours for strong hybridization signal) and RNAScope sold by Advance Cell Diagnostics (requiring at least 2 hours for strong hybridization signal), 30 to 60 min incubation with a simple hybridization buffer at 37° C. gives as strong binding signal as the other two methods with hours to overnight hybridization time, and also is comparable with the intensity by denaturing the sample at 70° C. for 10 min (in 80% formamide, 2×SSC buffer) and hybridizing at 37° C. for 5 min (in 10% formamide, 2×SSC buffer). An example image is shown in FIG. 8.

Rapid RNA FISH developed here required efficient heat denaturation with fast heat transfer rate. Typically, #1.5H glass coverslips (170 μm (+/−5 μm) in thickness, D 263 M Schott high precision glass) to hold biological samples to do heat denaturation on a stainless steel hot plate (Labnet Dry Bath, dual block. 120V). Two other materials were also tested for cell attachment and heat denaturation: #1.5 polymer coverslips (180 μm (+10/−5 μm) in thickness) sold by Ibidi and 1 mm glass slides. None of the latter two could do heat denaturation efficiently. It took 30-60 min to get a decent binding signal by polymer coverslips and 1 mm glass slides. However, even after such a long time of heat denaturation, the binding signal was still not as strong as 10 min heat denaturation at the same temperature with #1.5H glass coverslips and the non-specific binding was also much higher. This effect was tested and validated by three different cell lines: RPE, HeLa and MEF (mouse embryonic fibroblasts).

### Example 3

Rapid DNA FISH Requires Sufficient Heat Denaturation

To test how heat denaturation affects the hybridization speed of DNA FISH, a series of different heat denaturation conditions were tested in HeLa cells. HeLa cells were seeded into #1.5 glass bottom 8-well chambers (Ibidi) for heat denaturation and hybridization. Cells were fixed in 4% PFA for 5 min and then in pure methanol at room temperature for 5 min. The denature buffer was 80% formamide, 2×SSC. The hybridization buffer was 10% formamide, 2×SSC. 200 nM of oligo probes for the repetitive sequence of centromere used in Example 1 were used here. All experiments in this example were done with 5 min hybridization at 37° C.

The denaturation conditions tested here were as follows:

1. 70° C. 2 min;

2. 70° C. 5 min;

3. 75° C. 2 min; 70° C. 10 min;

4. 80° C. 10 min;

5. 95° C. 3 min.

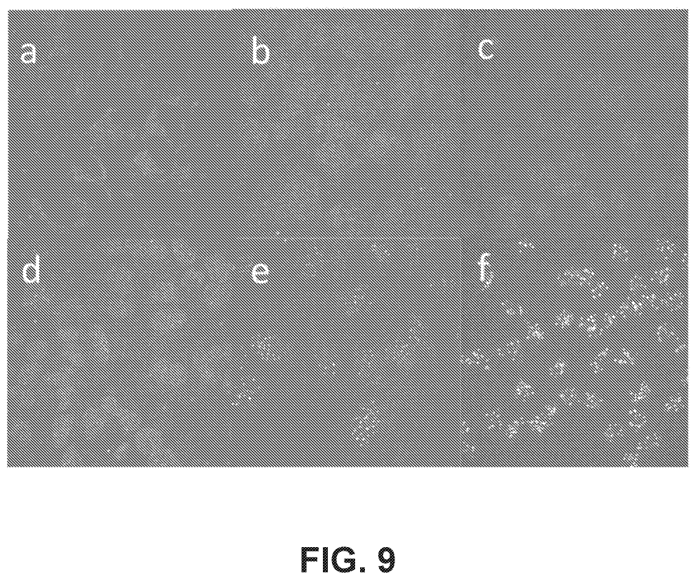

The images were shown in FIG. 9. As illustrated in FIG. 9, 2 min denaturation at 70° C. has no specific binding in the nucleus at all. Only autofluorescence mainly from the cytoplasm was observed. For 5 min denaturation at 70° C. or 2 min at 75° C., cells had some weak binding in the nucleus; while with 10 min denaturation at 70° C., some cells had good signal of centromere labeling but most of cells still had weak binding signal in the nucleus. With 10 min denaturation at 80° C., all the cells showed good binding signal of centromere in the nucleus. At higher temperature such as 95° C., only 3 min denaturation gave stronger binding signal than 10 min denaturation at 80° C. Therefore, sufficient heat denaturation and sufficient heating time resulted in rapid hybridization within 5 min at 37° C.

Traditional DNA FISH used a brief duration of heat denaturation (70° C.-75° C. for 2 min) to denature chromosomes in eukaryotic cells. This is not sufficient for rapid DNA FISH with a hybridization time less than 5 min in most of eukaryotic cells. Thorough heat denaturation with sufficient denaturation time and high temperature (>=70° C.) is necessary for the rapid FISH developed here. With the increase of denaturing temperature, both denaturing time and hybridization time could be shortened. When the denaturing temperature goes more than 80° C., short denaturation Lime (<5 min) is sufficient for rapid DNA FISH.

### Example 4

Rapid DNA FISH in Cultured Cells with a Hybridization Buffer Having Dextran Sulfate

A series of hybridization buffer (10% formamide and 2×SSC) with different concentration (5%, 10%, 15% and 20%, w/v) and molecular weight (5000 Da, 8000 Da, 15,000 Da, 40.000 Da and >500,000 Da) of dextran sulfate were tested in RPE cells. Cells were seeded into 8-well #1.5 coverglass bottom chambers for staining and imaging. The same short oligo probes for centromere and the repetitive sequence on chromosome 1 from Example 1 were used here. RPE cells were fixed in 4% PFA for 5 min and then in pure methanol at room temperature for 5 min. Cells were denatured at 80° C. in 80% formamide, 2×SSC for 10 min. The hybridization buffer used here was 10% formamide, 2×SSC with different concentration of dextran sulfate. Dextran sulfate with molecular weight less than 10,000 did not affect rapid DNA FISH positively or negatively, independent on its concentration. For dextran sulfate higher than 10,000 Da, lower than 10% neither affected the hybridization speed of rapid DNA FISH in a short time scale (less than 5 min) nor contributed to non-specific binding significantly. However, 10% and higher concentration increased non-specific binding significantly but didn't affect the hybridization speed of rapid DNA FISH. Therefore, dextran sulfate of high molecular weight, which was used as an acceleration agent for traditional DNA FISH, was not necessary for rapid DNA FISH developed here.

### Example 5

Rapid DNA FISH Requires Short Oligonucleotides:

2 DNA oligos with 100 nucleotides in length are tested using the same protocol developed in Example 1.

Sequence 1 for Telornere:
5′ ATGCCGAATGCTCTGGCCTCGAACGAACGATAGCCCCTAACCCTAA
CCCTAACCCTAACCCTAACCCTAACGCAACGCTTGGGACGGTTCCAATC
GGATC 3′
Sequence 2 for Centromere:
5′ ATGCATCAAGTATGCAGCGCGATTGACCGTCTCGTTATTCGTTGGA
AACGGGAATTCGTTGGAAACGGGAACAAATCCGACCAGATCGGACGATC
ATGGG

Sequence 1 contains 6 tandem repeats of CCCTAA which is complementary with the repetitive sequence of telomere in human.

Sequence 2 contains ATTCGTTGGAAACGGGA which is complementary with the repetitive sequence of centromere in human.

Both sequences were tested in RPE cells. Cells were seeded into 8-well #1.5 coverglass bottom chambers for staining and imaging. RPE cells were fixed in 4% PFA for 5 min and then in pure methanol at room temperature for 5 min. Cells were denatured at 80° C. in 80% formamide/2×SSC buffer for 10 min and then incubated with 100 nM oligo probes in 10% formamide/2×SSC hybridization buffer at 37° C. for 3-5 min. Both sequences can be delivered into the nucleus of RPE cells in 5 min at 37° C. Sequence 1 can detect telomere successfully but also has strong false positive binding in the cytoplasma of RPE cells. While sequence 2 completely lost the binding specificity to centromere and only non-specific binding in nucleus was observed. Therefore, longer sequences are not necessary to increase the binding specificity of DNA FISH but may decrease the hybridization speed significantly. Traditional DNA FISH uses bacterial artificial chromosome clones to synthesize oligonucleotide probes and then labels the probes by nick translation or random priming technology. This approach typically generates single strand DNA fragments with 200-1000 nucleotides in length, which takes much more time (hours to overnight) to hybridize with the targeted chromosome sequences. Typically, less than 100 nucleotides long oligo probes are used here for rapid DNA FISH, which is ½- 1/50 of the probe length used by traditional DNA FISH.

### Example 6

Rapid DNA FISH in Blood Cells

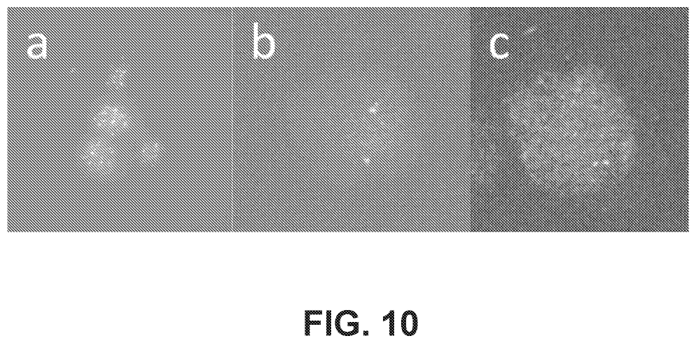

White blood cells isolated from peripheral blood were fixed in a combination of acetic acid and methanol solution (1:2 to 1:3). After fixation, cells were attached to either 1 mm frosted glass slides or #1.5 glass bottom 8-well chambers (Ibidi) and air dried. For repetitive sequences, cells were denatured at 80° C. for 5-10 min or 70° C. for 10-15 min in a denaturation buffer with 80% formamide. Then cells were hybridized with the same oligo probes used in Example 1 at 37° C. in a hybridization buffer with 10% formamide and 2×SSC. Individual FISH dots were observed after 2 min hybridization as shown in FIG. 10. For non-repetitive sequences, higher denaturation temperature may be required for thorough chromosome denaturation and rapid oligo binding. As shown in FIG. 10(c), an oligo pool library was synthesized and hybridized with the non-repetitive sequences on human chromosome 19 (NCBI human genome version 19, locus: 44720001-44820001). The library consists of 1096 oligos of different sequences and labeled with Atto647N. All oligos have 64-66 nucleotides in length and 45 nucleotides complementary with the targeted chromosome sequences. Blood cells were attached onto 1 mm glass slides here for non-repetitive sequence FISH. The denaturation condition was 90° C. for 5-10 min in 80% formamide, 2×SSC buffer. The hybridization condition is 100-200 nM probes in a buffer with 10% formamide and 2×SSC at 37° C. for 2 min.

For DNA FISH, both coverglasses and glass slides worked well but cells on coverglasses gave stronger signal and labeling efficiency at the same denaturation temperature due to more efficient heat transfer and less background, or required lower denaturation temperature to achieve the same hybridization efficiency.

Traditional DNA FISH used a brief duration of heat denaturation (70° C.-75° C. for 2 min) to denature chromosomes in blood cells. It takes hours to overnight to deliver long oligonucleotides (>200 nucleotides) to hybridize with the targeted sequences well. This short duration of heat denaturation was also not sufficient for rapid DNA FISH using short oligonucleotides (typically less than 100 nucleotides in length). Thorough heat denaturation with sufficient longer denaturation time and/or higher temperature (>75° C.) were necessary for rapid DNA FISH in blood cells. By extended heat denaturation and short oligonucleotide design, hybridization finishes in 1-2 min even with non-repetitive sequences.

### Example 7

Rapid DNA FISH in Mouse Fresh Frozen Tissues

Mouse liver and lung fresh frozen tissues were used here to do DNA FISH. Tissues were warmed to room temperature and air dried first. After that, they were fixed in 4% PFA 5 min first and then in pure methanol at room temperature for 5 min. The same oligo probes to target the repetitive sequences for telomere and centromere in Example 1 are used here as these sequences are shared by mouse and human. The same protocol of rapid DNA FISH developed for culture cell lines could be used here after tissue fixation. Typically, a tissue sample was denatured at 80° C. for 10-15 min in 80% formamide/2×SSC buffer and then hybridized with 100-200 nM oligo probes at 37° C. for 3-5 min. Fixation and permeabilization could last longer (from minutes to hours or even overnight) or using higher concentration of PFA. However, with longer and stronger fixation, longer denaturing time (>15 min) and/or denaturing temperature were required to denature the chromosome DNA thoroughly for rapid FISH developed here.

### Example 8

Rapid DNA FISH in Mouse Fixed Frozen Tissues:

Mouse primary tissues were freshly harvested from CD1 mice, fixed in 10% neutral buffered formalin overnight, cryoprotected in 30% sucrose, and snapped frozen in O.C.T. freezing medium. Frozen tissues were cut at a thickness of 10 um and mounted on slides treated for adherence.

Frozen tissues were warmed up to room temperature for at least 30 min. After that, tissues were washed by PBS buffer 2× and 2×SSC buffer once. Then tissues were incubated with denaturation buffer (80% formamide and 2×SSC) at 80° C. The same oligo probe targeting the repetitive sequence of centromere in human in Example 1 is used here in mouse as this repetitive sequence is shared by the two species. Due to overnight fixation, normal denaturation time (5-10 min) for cultures cells in Example 1 didn't work well for fixed frozen tissues. Extending the denaturation time to 15-20 min gave stronger signal and higher labeling efficiency. Meanwhile, hybridization still finished in 5 min at 37° C. with the same hybridization buffer (10% formamide, 2×SSC).

Rapid FISH developed here is compatible with signal amplification techniques such as tyramide labeling, branched DNA amplification and hybridization chain reaction (BioTechniques 27:608-613, ACS Nano. 8(5): 4284-4294) for final signal detection and imaging. This is quite useful when the samples generate high background such as the fixed frozen primary tissues used here. Taking tyramide signal amplification as an example. Oligonucleotides can be labeled with biotin to facilitate tyramide signal amplification. The labeled oligo probes are hybridized with the samples by rapid DNA/RNA FISH developed here at first and then the samples are incubated with horseradish peroxidase conjugated streptavidin and in the dye-tyramide working solution to allow tyramide deposition. In this way, the samples are ready for imaging.

### Example 9

Rapid DNA and RNA FISH in Bacteria:

Rapid DNA and RNA FISH developed here can also be used in prokaryotic cells such as bacteria and other microbes to detect chromosome and RNA sequence in prokaryotic cells. Here is an example. Oligonucleotide probes with each oligo less than 100 nucleotides in length are designed and synthesized based on the targeted DNA or RNA sequences in prokaryotic cells. Sample preparation and staining are done as follows: Add formaldehyde to a bacterial culture sample (e.g. E. coli) to a final concentration of 2% (v/v). Fix the cells for 0.5-1 h at room temperature. Centrifuge the sample at 16,000 g for 10 min and resuspend in PBS. Repeat twice. Mix 100 ul sediment with 900 ul PBS and sonicate with a sonication probe at minimum power for 20 sec. Mix the sonicated sample with 0.1% agarose in PBS containing 0.001% SDS. Incubate at 55° C. for 5 min. Pipette 10 ul of the sample suspension onto the 1 mm glass slides or 0.17 mm glass bottom 8-well chambers. Allow to dry at room temperature. Permeabilize cell walls as required for the sample of interest using either lysozyme or proteinase K (0.1-10 ug/ml) for 30-60 min at 37° C. Denature the prepared sample slides or chambers in 80% formamide, 2×SSC at 80-90° C. for 5-10 min. The actual temperature and duration of heat denaturation can be adjusted according to the targeted sequences for thorough denaturation. Hybridize the samples with 100-200 nM oligonucleotide probe hybridization mix at 37° C. for 3-5 min in 80% formamide, 2×SSC. Wash the samples 2× in 2×SSC buffer. Stain the samples with DAPI and then the samples are ready for imaging. This protocol works for both rapid DNA and RNA FISH in bacteria.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

1-17. (canceled)

18. A method of in situ RNA hybridization, wherein the method comprises,
denaturing RNA sequences in fixed cells at a temperature above 37° C.:
contacting the cells with a first single-stranded oligonucleotide under conditions for the first oligonucleotide to hybridize with a first RNA sequence among the RNA sequences;
detecting the first oligonucleotide hybridized to the first RNA sequence in the cells.

19. The method of claim 18, further comprising:
removing the first oligonucleotide hybridized to the first RNA sequence; and then
contacting the cells with a second single-stranded oligonucleotide under conditions for the second oligonucleotide to hybridize with a second RNA sequence among the RNA sequences;
and
detecting the second oligonucleotide hybridized to the second RNA sequence in the cells.

20. (canceled)

21. The method of claim 18, wherein the denaturing is carried out in the presence of a nucleic acid strand interaction-weakening agent.

22. (canceled)

23. The method of claim 21, wherein the nucleic acid strand interaction-weakening agent is formamide or ethylene carbonate.

24. (canceled)

25. The method of claim 23, wherein the denaturing is carried out in the presence of 70-95% formamide for at least 5 or 10 minutes.

26. The method of claim 18, wherein the method takes less than 45 minutes to complete.

27. The method of claim 18, wherein the contacting takes less than 10 minutes.

28. The method of claim 18, wherein the cells are not contacted with more than 0.1% dextran sulfate.

29. The method of claim 18, wherein the cells are not contacted with dextran sulfate.

30. The method claim 18, wherein the first oligonucleotide is fluorescent labeled.

31. The method of claim 18, wherein the contacting is carried out in the presence of formamide for less than 10 minutes.

32-34. (canceled)

35. The method of claim 18, wherein the first single-stranded oligonucleotide is 15-35 nucleotides long.

36. The method of claim 18, wherein the cells are mammalian cells.

37. The method of claim 18, wherein the denaturing is carried out at a temperature of 50° C. or higher.

38. The method of claim 18, wherein the denaturing is carried out at a temperature of 70° C. or higher.

39. The method of claim 18, wherein the denaturing lasts less than 20 min.

40. The method of claim 18, wherein the cells are fixed on a glass slide having a thickness of 1 mm or less before denaturing.

41. The method of claim 40, wherein the glass slide has a thickness of about 0.17 mm.

42. The method of claim 18, wherein the contacting is carried out in a solution in which none components at a concentration of at least 5% have a molecular weight of greater than 1000 Daltons.

43. The method of claim 18, wherein the first oligonucleotide is shorter than 100 nucleotides.

44. The method of claim 18, wherein the first oligonucleotide is shorter than 50 nucleotides.

45. The method of claim 19, wherein the first oligonucleotide that is hybridized to the first RNA sequence is removed by heating the cells at a temperature of 50° C. or higher.


SEQUENCE LISTING (14 sequences)

SEQ ID NO: 1 - RNA, 22 nt, Artificial Sequence
  Description of Artificial Sequence Synthetic oligonucleotide
guggcgugaccuguggaugcug

SEQ ID NO: 2 - RNA, 19 nt, Artificial Sequence
  Description of Artificial Sequence Synthetic oligonucleotide
gcucuuauggugagagugu

SEQ ID NO: 3 - RNA, 22 nt, Artificial Sequence
  Description of Artificial Sequence Synthetic oligonucleotide
gaaggaaugguccaugcuuacc

SEQ ID NO: 4 - RNA, 21 nt, Artificial Sequence
  Description of Artificial Sequence Synthetic oligonucleotide
ggcaaggcaaggcaaggcaca

SEQ ID NO: 5 - DNA, 21 bp, Artificial Sequence
  Description of Artificial Sequence Synthetic probe
ccaggtgagcatctgacagcc

SEQ ID NO: 6 - DNA, 15 bp, Artificial Sequence
  Description of Artificial Sequence Synthetic probe
cttcctgtcaccgac

SEQ ID NO: 7 - DNA, 22 bp, Artificial Sequence
  Description of Artificial Sequence Synthetic probe
ccactgtgatatcatacagagg

SEQ ID NO: 8 - DNA, 22 bp, Artificial Sequence
  Description of Artificial Sequence Synthetic probe
cccacactctcaccataagagc

SEQ ID NO: 9 - DNA, 22 bp, Artificial Sequence
  Description of Artificial Sequence Synthetic probe
ggtaagcatggaccattccttc

SEQ ID NO: 10 - DNA, 21 bp, Artificial Sequence
  Description of Artificial Sequence Synthetic probe
ttgccttgtgccttgccttgc

SEQ ID NO: 11 - DNA, 18 bp, Artificial Sequence
  Description of Artificial Sequence Synthetic probe
ccctaaccctaaccctaa

SEQ ID NO: 12 - DNA, 17 bp, Artificial Sequence
  Description of Artificial Sequence Synthetic probe
attcgttggaaacggga

SEQ ID NO: 13 - DNA, 100 bp, Artificial Sequence
  Description of Artificial Sequence Synthetic polynucleotide
atgccgaatgctctggcctcgaacgaacgatagcccctaaccctaaccctaaccctaaccctaaccctaacgcaacgcttgggacggttccaatcggatc

SEQ ID NO: 14 - DNA, 100 bp, Artificial Sequence
  Description of Artificial Sequence Synthetic polynucleotide
atgcatcaagtatgcagcgcgattgaccgtctcgttattcgttggaaacgggaattcgttggaaacgggaacaaatccgaccagatcggacgatcatggg